(19) United States
(12) Patent Application Publication
(10) Pub. No.: US 2007/0260039 A1
(43) Pub. Date: Nov. 8, 2007

(54) Methods of Producing Silk Polypeptides and Products Thereof

(76) Inventors: Costas N. Karatzas, Beaconsfield (CA); Carl Turcotte, Monteal (CA)

Correspondence Address: JONES DAY, 222 EAST 41ST ST, NEW YORK, NY, 10017, US

(21) Appl. No.: 10/501,183

(22) PCT Filed: Jan. 13, 2003
(86) PCT No.: PCT/IB03/00346

§ 371 Date: Oct. 7, 2006

Related U.S. Application Data
(60) Provisional application No. 60/347,509, filed on Jan. 11, 2002.

(51) Int. Cl. C07K 14/00; C07K 5/00; C07K 7/00

(52) U.S. Cl. 530/324; 530/325; 530/326; 530/327; 530/328; 530/329; 530/330; 530/331; 530/353

(57) ABSTRACT

The invention provides a silk polypeptide comprising a plurality of repetitive units and a non-repetitive hydrophilic amino acid domain, polynucleotides and vectors encoding silk polypeptides, methods of expressing the silk polypeptide in host cells and transgenic animals, and methods of forming a biofilament comprised of silkpolypeptides.

This application is entitled to and claims priority benefit under 35 U.S.C. § 119(e) to U.S. Provisional Applications No. 60/347,509, filed Jan. 11, 2002, incorporated herein by reference in its entirety.

### 1. FIELD OF THE INVENTION

The present invention relates to the expression of silk polypeptides in host cells and transgenic animals.

### 2. BACKGROUND OF THE INVENTION

The silks of spiders and lepidopteran insects are proteinaceous fibers, or biofilaments, composed largely of non-essential amino acids. Orb-web spinning spiders have as many as seven sets of highly specialized glands and produce up to seven different types of silk. Each silk fiber has a different amino acid composition, mechanical property and function. The physical properties of a silk fiber are influenced by the amino acid sequence, spinning mechanism, and environmental conditions in which it was produced.

Native spider silk polypeptides are designated according to the gland or organ of the spider in which they are produced. Spider silks known to exist include major ampullate (MaSp), minor ampullate (MiSp), flagelliform (Flag), tubuliform, aggregate, aciniform, and pyriform spider silk proteins. Spider silk proteins derived from each organ are generally distinguishable from those derived from other synthetic organs by virtue of their physical and chemical properties. For example, major ampullate silk, or dragline silk, is extremely tough. Minor ampullate silk, used in web construction, has high tensile strength. An orb-web's capture spiral, in part composed of flagelliform silk, is elastic and can triple in length before breaking. Gosline et al., J. Exp. Biol. 202:3295 (1999). Tubuliform silk is used in the outer layers of egg-sacs, whereas aciniform silk is involved in wrapping prey, and pyriform silk is laid down as the attachment disk.

Dragline silk is one of the strongest silks studied and possesses unique mechanical properties suitable for technical applications. The protein forming the core of dragline silk fibers is secreted as a mixture of two soluble proteins from specialized columnar epithelial cells of the major ampullate gland of orb-weaver spinning spiders. The dragline silk of Araneus diadematus demonstrates high tensile strength (1.9 Gpa; ˜15 gpd) approximately equivalent to that of steel (1.3 Gpa) and aramid fibers. The physical properties of dragline silk balance stiffness and strength both in extension and compression imparting the ability to dissipate kinetic energy without structural failure.

The utility of spider silk proteins as “super filaments” has led to attempts to produce recombinant spider dragline silks in bacterial and yeast systems with moderate success (Kaplan et al., Mater. Res. Soc. Bull. 10:41-47 (1992); Fahnestock & Irwin, Appl. Microbiol. Biotechnol. 47:23-32 (1997); Prince, Biochemistry 34:10879-10885 (1995); Fahnestock & Bedzyk, Appl. Microbiol. Biotechnol. 47:33-39 (1997)). However, the recombinant proteins expressed to date have not resulted in useful biofilaments, as the fibers spun from these recombinant proteins are brittle, which may be due to smaller size of the expressed and purified recombinant proteins as compared to natural occurring silk proteins.

Part of the technical challenge is overcoming the difficulty expressing silk proteins due to the highly repetitive structure and the unusual secondary structure at the mRNA level, which leads to inefficient translation due to pausing and to premature termination of synthesis, thus limiting the length of the silk polypeptide produced (Hinman et al., Trends in Biotech. 18:374-379 (2000)). It has been further demonstrated that spider silk genes are unstable due to recombination and rearrangement in the repetitive areas of the gene. As a result, successful expression of recombinant spider silk genes in E. coli has been limited to a protein of 43-58 kDa (Lewis et al., Protein. Expr. Purif. 7:400-405 (1996); Arcidiacono et al., Appl. Microbiol. Biotechnol. 49:31-38 (1998)). Expression of silk polypeptides larger than those produced in E. coli have been reported in the methylotropic yeast Pichia pastoris and transgenic plants, but biofilaments formed from such silk polypeptides have not been reported to possess useful physical properties, possibly due to solubility difficulties. (Fahnestock et al., Reviews Mol. Biotech. 74:105-119 (2000); Scheller et al., Nature Biotech. 19:573-577 (2001)).

Thus, there remains an unmet need for silk polypeptides, and methods of producing such silk polypeptides, that can be used to make biofilaments having useful properties similar to those of natural spider and lepidopteran insect silks, such as strength and elasticity.

### 3. SUMMARY OF THE INVENTION

The present invention presents isolated silk polypeptides, methods of producing isolated silk polypeptides, and methods of producing biofilaments having properties similar or superior to those of naturally occurring spider and insect silks. Thus, in certain aspects, the invention provides isolated silk polypeptides comprising a plurality of repetitive units and a non-repetitive hydrophilic amino acid domain, wherein the isolated silk polypeptide has a molecular weight ranging from about 16 kDa to about 800 kDa. In other embodiments, the isolated silk polypeptide has a molecular weight within the range of 58 kDa to 800 kDa. In other embodiments, the isolated polypeptide has a molecular weight between about 55 kDa to about 100 kDa. In additional embodiments, the silk polypeptide has a molecular weight in the ranges of about 100 kDa to about 300 kDa, and about 300 to about 800 kDa.

In certain embodiments, the invention further provides isolated silk polypeptides wherein at least two of the repetitive units are placed in a head-to-head configuration. In further embodiments, the invention provides isolated silk polypeptides wherein the repetitive units are placed in a head-to-head configuration. In other embodiments, the invention provides isolated silk polypeptides wherein at least two of the repetitive units are placed in a head-to-tail configuration. In further embodiments, the invention provides isolated silk polypeptides wherein the repetitive units are placed in a head-to-tail configuration. In certain embodiments, the isolated silk polypeptides comprise at least about 2 to about 4 repetitive units. In other embodiments, the isolated silk polypeptides comprise at least about 5 to about 10 repetitive units. In still other embodiments, the isolated silk polypeptides comprise at least about 10 to about 50 repetitive units. In yet other embodiments, the isolated silk polypeptides comprise at least about 100 to about 1000 repetitive units.

In certain embodiments, the invention provides isolated silk polypeptides comprising a plurality of repetitive units and a non-repetitive hydrophilic amino acid domain, wherein at least two of the repetitive units are contiguous. In certain embodiments, each of the repetitive units are contiguous. In other embodiments, at least two of the repetitive units are separated by an amino acid spacer. In certain embodiments, each of the repetitive units is separated from each other by an amino acid spacer. In certain embodiments, the amino acid spacer is between 1 amino acid to about 10 amino acids in length.

In other aspects, the invention provides isolated silk polypeptides comprising a plurality of repetitive units and a non-repetitive hydrophilic amino acid domain, wherein the repetitive units comprise amino acid sequences that form secondary structures selected from the group consisting of: β-turn spiral, crystalline β sheet, and 3₁₀ helix. In other embodiments, the invention provides isolated silk polypeptides comprising a plurality of repetitive units and a non-repetitive hydrophilic amino acid domain, wherein the repetitive units comprise a combination of amino acid sequences that form secondary structures selected from the group consisting of: β-turn spiral, crystalline β sheet, and 3₁₀ helix. In certain embodiments, the repetitive units comprise a repetitive unit found within a spider or insect silk polypeptide. In other embodiments, each repetitive unit independently comprises a repetitive unit found within Nephila clavipes or Araneus diadematus spider silk polypeptides or Bombyx mori cocoon silk polypeptides. In yet other embodiments, the amino acid sequence of each repetitive unit can be independently selected from the group consisting of amino acid sequences of ADF-1, ADF-2, ADF-3, ADF-4, ABF-1, MaSpI MaSpII, MiSpI, MiSpII, and Flag. In a preferred embodiment, the amino acid sequence of each repetitive unit is selected from the group consisting of the amino acid sequences of SEQ ID NOS:1-3, as shown in FIGS. 5, 6 and 7, respectively. In yet other embodiments, at least one of the repetitive units can have an amino acid sequence that is in a reversed order in comparison to the naturally-occurring amino terminus to carboxyl terminus amino acid sequence. In still other embodiments, the repetitive units comprise iterated peptide motifs selected from the group consisting of the amino acid sequences identified as SEQ ID NOS:4-27. In still other embodiments, the repetitive units comprise repetitive units forming an amorphous domain and a crystal-forming domains. Preferably, such repetitive units comprise amino acid sequences identified as SEQ ID NO:28 and SEQ ID NO:29. In still other embodiments, the repetitive units comprise a plurality of iterated peptide motifs selected from the group consisting of: GPG(X)ₙ, (GA)ₙ, Aₙ, and GGX, wherein X represents the amino acid A, Q, G, L, S, Y or V, and n represents an integer from 1 to about 8. In still other embodiments, at least two of the repetitive units have identical amino acid sequences. In yet other embodiments, the repetitive units have identical amino acid sequences. In still other embodiments, at least two repetitive units can have non-identical amino acid sequences.

In other aspects, the invention provides isolated silk polypeptides comprising a plurality of repetitive units and a non-repetitive hydrophilic amino acid domain, wherein the non-repetitive hydrophilic amino acid domain can be toward the carboxyl terminus with respect to the repetitive units. In other embodiments, the non-repetitive hydrophilic amino acid domain can be toward the amino terminus with respect to the repetitive units. In yet other embodiments, the non-repetitive hydrophilic amino acid domain can be between two of the repetitive units. In other aspects, the invention further provides isolated silk polypeptides having a plurality of repetitive units and a non-repetitive hydrophilic amino acid domain, further comprising a proteolytic site, wherein cleavage at the proteolytic site cleaves the non-repetitive hydrophilic amino acid domain from a repetitive unit. In other embodiments, the invention further provides isolated silk polypeptides having a plurality of repetitive units and a non-repetitive hydrophilic amino acid domain, further comprising a first proteolytic site and a second proteolytic site, wherein cleavage at the first proteolytic site and at the second proteolytic site cleaves the non-repetitive hydrophilic amino acid domain from the repetitive units.

In still other aspects, the invention provides isolated silk polypeptides having a plurality of repetitive units and a non-repetitive hydrophilic amino acid domain, wherein the non-repetitive hydrophilic amino acid domain can have an amino acid sequence that is identical or substantially identical to sequences selected from the group consisting of amino acid sequences of non-repetitive hydrophilic carboxyl terminal regions of MaSpI, MaSpII, MiSpI, MiSpII, ABF-1, ADF-1, ADF-2, ADF-3, ADF-4, NCF-1, NCF-2, and Flag. In certain embodiments, the non-repetitive hydrophilic amino acid domain can be about 20 to about 150 amino acids in length.

In yet other aspects, the invention provides isolated silk polypeptides having a plurality of repetitive units and a non-repetitive hydrophilic amino acid domain, further comprising one or more additional non-repetitive hydrophilic amino acid domains. In certain embodiments, the one or more additional non-repetitive hydrophilic amino acid domains comprises at least about 2 to about 4 non-repetitive hydrophilic amino acid domains.

In certain embodiments, the isolated silk polypeptides further comprise a proteolytic site, wherein cleavage at the proteolytic site results in the separation of all, substantially all, or a portion of the non-repetitive hydrophilic amino acid domain from a repetitive unit. In certain embodiments, the isolated silk polypeptides further comprise a proteolytic site, wherein cleavage at the proteolytic site results in the separation of all, substantially all, or a portion of the non-repetitive hydrophilic amino acid domain from the repetitive units. In other embodiments, the isolated silk polypeptides further comprise a first proteolytic site and a second proteolytic site, wherein cleavage at the first proteolytic site and at the second proteolytic site cleaves all, substantially all, or a portion of the non-repetitive hydrophilic amino acid domain from the repetitive units. In still other embodiments the non-repetitive hydrophilic domain can contain a proteolytic site that can be located such that cleavage at the proteolytic site can remove the non-repetitive hydrophilic amino acid domain from the non-repetitive units.

In certain embodiments, all, substantially all, or a portion of the non-repetitive hydrophilic amino acid domain can be cleaved from the repetitive units endogenously within the expression system before purification of the silk polypeptides. In further embodiments, all, substantially all, or a portion of the non-repetitive hydrophilic amino acid domain can be cleaved from the repetitive units before, during, or after secretion of the silk polypeptides into a biological fluid, including milk of a lactating female mammal or urine, before purification of the silk polypeptides. In other embodiments, all, substantially all, or a portion of the non-repetitive hydrophilic amino acid domain can be cleaved from the repetitive units following purification of the silk polypeptides. In certain embodiments, the proteolytic site is subject to cleavage by a protease. In other embodiments, the proteolytic site is subject to cleavage by chemical treatment.

In certain embodiments, the isolated silk polypeptides of the invention further comprise a secretory signal peptide sequence. In certain embodiments, the isolated silk polypeptides of the invention further comprise a c-myc epitope. In other embodiments, the isolated silk polypeptides of the invention further comprise a histidine tag.

In still other aspects, the invention provides isolated silk polypeptides having a plurality of repetitive units and a non-repetitive hydrophilic amino acid domain, wherein the silk polypeptide precipitates and redissolves in an aqueous buffer.

In other aspects, the invention provides isolated polynucleotides encoding the silk polypeptides of the invention. In certain embodiments, invention provides isolated polynucleotides comprising a nucleotide sequence encoding more than one repetitive unit in a single open reading frame, wherein the repetitive units are independently selected from the group consisting of repetitive units of ADF-1, ADF-2, ADF-3, ADF-4, ABF-1, MaSpI, MaSpII, MiSpI, MiSpII, and Flag. In certain embodiments, the polynucleotide encodes an silk polypeptide of the invention, wherein the repeat units are encoded in their native 5′ to 3′ direction.

In yet other aspects, the invention further provides vectors comprising the polynucleotides of the invention. In certain embodiments, the vector can be an expression vector further comprising a promoter, wherein the promoter is operably linked to the coding sequence of a silk polypeptide of the invention. In certain embodiments, the promoter can be a tissue-specific promoter selected from the group consisting of uromodulin promoter, uroplakin I, II, and III promoters, rennin promoter, WAP promoter, β-casein promoter, αS1-casein promoter, αS2-casein promoter, κ-casein promoter, β-lactoglobin, and α-lactalbumin promoter. In certain embodiments, the expression vector can further comprise a leader sequence that enables secretion of the biofilament protein by cells transformed or transfected with the expression vector.

In still other aspects, the invention provides a host cell transformed or transfected with an expression vector of the invention. In yet other aspects, the invention provides a method of producing the silk polypeptides of the invention, comprising culturing a host cell containing a polynucleotide encoding a silk polypeptide of the invention under conditions that cause the host cell to express the silk polypeptide, and purifying the silk polypeptide from the host cell or from the cell culture media. In certain embodiments, the host cell can be a prokaryotic host cell. In other embodiments, the host cell can be a eukaryotic host cell. In further embodiments, the host cell can be a plant host cell. In still further embodiments, the host cell can be a yeast host cell. In yet further embodiments, the host cell can be a mammalian host cell. In still further embodiments, the mammalian host cell can be a mammalian epithelial cell. In still further embodiments, the mammalian epithelial cell can be a MAC-T cell or a BHK cell. In certain embodiments, the host cell can constitutively secrete a silk polypeptide of the invention. In certain embodiments, the host cell can have a polynucleotide integrated into its genome, wherein the polynucleotide encodes a silk polypeptide of the invention. In certain embodiments, the host cell further comprises a polynucleotide encoding a protease. In further embodiments, the protease can be native to the host cell. In other embodiments, the protease can be non-native to the host cell. In certain embodiments, the host cell can co-express a plurality of the silk polypeptides of the invention.

In yet other aspects, the invention provides a non-human transgenic mammal that secretes into its urine a silk polypeptide of the invention. In certain embodiments, the non-human transgenic mammal can be a ruminant. In further embodiments, the non-human transgenic mammal can be a goat. In other embodiments, the invention provides a non-human lactating female transgenic mammal that expresses in its milk a silk polypeptide of the invention. In certain embodiments, the non-human lactating female transgenic mammal can be a ruminant. In further embodiments, the non-human lactating female transgenic mammal can be a goat. In certain embodiments, the lactating female goat can express in its milk a silk polypeptide that comprises a proteolytic site, wherein the proteolytic cleavage occurs before the silk polypeptide is purified from the milk.

In certain embodiments, the silk polypeptide that is made according to the methods of the invention can further comprise a proteolytic site, wherein cleavage at the proteolytic site cleaves all, substantially all, or a portion of the non-repetitive hydrophilic amino acid domain from the repetitive units. In certain embodiments, the nucleic acid encoding the silk polypeptide can be operably linked to a regulatory sequence for expression of the silk polypeptide, wherein the regulatory sequence comprises a promoter. In certain embodiments, the promoter can be inducible, for example, by a developmental stage. In other embodiments, the promoter can be cell-type specific, for example, for a milk-producing cell or a urine-producing cell.

In other aspects, the invention further provides a method of producing the silk polypeptides of the invention, comprising expressing a silk polypeptide of the invention in a transgenic non-human animal and recovering the silk polypeptide from a biological fluid produced by the transgenic animal. In certain embodiments, the non-human transgenic animal can be a female mammal and the biological fluid can be milk. In other embodiments, the biological fluid can be urine. In other embodiments the biological fluid can be blood. In still other embodiments, the biological fluid can be saliva. In certain embodiments, the silk polypeptide according to the methods of the invention further comprises a proteolytic site, wherein cleavage at the proteolytic site cleaves the non-repetitive hydrophilic amino acid domain from the repetitive units. In further embodiments, cleavage at the proteolytic site can occur in the mammal before recovery of the portion of the silk polypeptide that corresponds to the repetitive units.

In yet other aspects, the invention provides a method of producing an isolated silk polypeptide for use in forming a biofilament, comprising purifying a polynucleotide encoding a silk polypeptide, wherein the silk polypeptide comprises a plurality of repetitive units and a non-repetitive hydrophilic amino acid domain, and wherein the silk polypeptide has a molecular weight between about 58 kDa and about 800 kDa; and expressing the polynucleotide in a host cell or transgenic mammal, Wherein the host cell expresses the silk polypeptide or the transgenic mammal secretes the silk polypeptide into a biological fluid.

In still other aspects, the invention provides a method for producing a silk polypeptides of the invention in a biological fluid of a transgenic animal, comprising introducing a nucleic acid molecule in a zygote, or embryo or cell line (for example, fetal fibroblast or adult somatic cell) to be used in nuclear transfer experiments wherein the nucleic acid molecule comprises a nucleic acid sequence encoding the silk polypeptide, a promoter that directs expression of the polypeptide in milk-producing cells or urine-producing cells or seminal fluid or saliva of an animal, in which the promoter is operably linked to the nucleic acid sequence, and a leader sequence that enables secretion of the silk polypeptide by the milk-producing cells or the urine-producing cells or seminal fluid-producing cells or saliva-producing cells into milk or urine or seminal fluid or saliva, respectively, of the animal; implanting the resulting genetically modified embryo (result of, for example, microinjection or nuclear transfer) or zygote into a recipient animal for gestation and birth; and recovering the silk polypeptide from the biological fluid of the transgenic animal that develops from the genetically engineered embryo. In certain embodiments, the nucleic acid sequence encodes a silk polypeptide as described herein. In certain embodiments, the leader sequence comprises an Ig-kappa leader sequence. In certain embodiments, the transgenic animal can be selected from the group consisting of a cow, a goat, a sheep, and a pig.

In other aspects, the invention provides methods of producing a biofilament composed of a plurality of one or more isolated silk polypeptides, comprising culturing a host cell that expresses the plurality of one or more silk polypeptides; purifying the plurality of one or more silk polypeptide; and spinning the plurality of one or more silk polypeptide to form a biofilament. In certain embodiments, the plurality of silk polypeptide comprises a proteolytic site. In certain embodiments, the plurality of silk polypeptides can be of 8 to 1,000 silk polypeptides.

In other aspects, the invention provides methods of producing a biofilament composed of a plurality of one or more isolated silk polypeptides, comprising expressing the plurality of one or more silk polypeptides in a transgenic plant or non-human mammal, purifying the plurality of one or more silk polypeptides from a plant extract or exudate or from a biological fluid of the non-human mammal; and spinning the plurality of one or more silk polypeptide to form a biofilament. In certain embodiments, the plurality of silk polypeptides comprise a proteolytic site. In certain embodiments, the plurality of silk polypeptides can be of 8 to 1,000 silk polypeptides.

In still other aspects, the invention provides a method of producing a biofilament, comprising expressing in a host cell or transgenic animal a silk polypeptide comprising a plurality of repetitive units, a non-repetitive hydrophilic amino acid domain, and a proteolytic site operably linked to the non-repetitive hydrophilic amino acid domain such that cleavage at the proteolytic site results in separation of the non-repetitive hydrophilic amino acid domain from the plurality of repetitive units; purifying the silk polypeptide; and spinning the biofilament from a solution comprising a portion of the silk polypeptide remaining after the non-repetitive hydrophilic amino acid domain has been removed by cleavage at the proteolytic site. In certain embodiments, the non-repetitive hydrophilic amino acid domain can be cleaved from the plurality of repetitive units in the host cell or transgenic animal. In certain embodiments, the silk polypeptide has a molecular weight between about 55,000 daltons and about 800,000 daltons. In other embodiments, the method of producing a biofilament can additionally comprise the step of cleaving the non-repetitive hydrophilic amino acid domain from the plurality of repetitive units.

In other aspects, the invention provides biofilaments produced according to the methods of the invention. In certain embodiments, the biofilaments can have a toughness between about 0.6 gpd and about 1.4 gpd. In certain embodiments, the biofilament can have a tenacity of between about 1.7 gpd and about 8.0 gpd.

### 4. TERMINOLOGY

“Biofilament,” as used herein, refers to a fibrous polymeric protein composed of silk polypeptides, including recombinantly-produced spider or insect silk monomers. Biofilaments are composed of alternating crystalline and amorphous regions. Exemplary biofilaments include spider silk, an externally spun proteinaceous fibrous secretion produced by a variety of spiders (e.g., Nephila clavipes), and fibroin, an externally spun proteinaceous fibrous secretion produced by in a variety of lepidopteran insects (e.g., Bombyx mori). Desirable biofilaments, when subjected to shear forces and mechanical extension during secretion, have a poly-alanine segment that undergoes a helix to β-sheet transition during such secretion, thereby forming a stable β-sheet crystal-forming structure. Desirably, the crystal-forming region of a silk polypeptide forms a β-pleated sheet such that inter-β-sheet spacings are between about 3 angstroms and about 8 angstroms in size, desirably, between about 3.5 angstroms and about 7.5 angstroms in size.

“Dope solution,” as used herein, refers to any liquid mixture that contains silk protein and is amenable to extrusion for the formation of a biofilament.

“Toughness,” as used herein, refers to the energy needed to break the biofilament, expressed as grams per denier (gpd). This energy can be calculated from the area under the force elongation curve, and is sometimes referred to as “energy to break” or “work to rupture.”

“Spinning,” as used herein, refers to the process of making a biofilament by extrusion, drawing, twisting, or winding silk polypeptides.

“Tenacity” or “tensile strength,” as used herein, refers to the amount of weight a biofilament can bear before breaking.

“Isolated silk polypeptide,” as used herein, refers to a silk polypeptide or protein (it is noted that, unless otherwise indicated, these two terms, as used herein, are interchangeable) that is expressed in an recombinant (e.g., microbial, plant or mammalian) expression system, i.e., separate from its natural milieu. “Isolated silk polypeptide” does not encompass silk polypeptides as found in their natural source. Nor are the isolated silk polypeptides of the invention ones that constitute native polypeptides purified from a natural source. In particular, an “isolated” or “purified” silk polypeptide is substantially free of cellular material or other contaminating proteins from the cell or tissue source from which the protein is derived. The language “substantially free of cellular material” includes preparations of a silk polypeptide in which the silk polypeptide is separated from cellular components of the cells from which it is recombinantly produced. Thus, a silk polypeptide that is substantially free of cellular material includes preparations of silk polypeptide having less than about 30%, 20%, 10%, or 5% (by dry weight) of contaminating protein. When the a silk polypeptide is expressed in cell culture, it is also preferably substantially free of culture medium, i.e., culture medium represents less than about 20%, 10%, or 5% of the volume of the protein preparation. In a preferred embodiment of the present invention, silk polypeptides are isolated or purified.

An “isolated” nucleic acid molecule or polynucleotide (it is noted that, unless otherwise indicated, these two terms, as used herein, are interchangeable) is one which is separated from other nucleic acid molecules or polynucleotides which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. An “isolated” nucleic acid molecule does not include cDNA molecules within a cDNA library. In a preferred embodiment of the invention, nucleic acid molecules encoding antibodies are isolated or purified. In another preferred embodiment of the invention, nucleic acid molecules encoding silk polypeptides are isolated or purified.

The term “host cell” as used herein refers to the particular subject cell transfected with a nucleic acid molecule or infected with phagemid or bacteriophage and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

“Transgene,” as used herein, refers to any piece of nucleic acid that is inserted by artifice into a cell or embryo, or an ancestor thereof, and preferably becomes part of the genome of the animal which develops from that cell. Such a transgene may include a gene which is partly or entirely heterologous (i.e., foreign) to the transgenic animal, or may represent a gene homologous to an endogenous gene of the animal. Such a transgene may also contain two or more gene sequences operably linked.

“Transgenic,” as used herein, refers to any cell which includes a nucleic acid sequence that has been inserted by artifice into a cell or embryo, or an ancestor thereof, and becomes part of the genome of the animal which develops from that cell. Preferably, the transgenic animals are transgenic mammals (e.g., rodents or ruminants). Desirably the nucleic acid (transgene) is inserted by artifice into the nuclear genome.

“Head-to-tail” and “head-to-head” as used herein, refers to the orientation of two or more repetitive units linked together within a silk polypeptide, or as encoded for by a polynucleotide. When repetitive units are in a head-to-tail orientation, each repetitive unit has a sequence that corresponds to the ordinary N-terminus to C-terminus amino acid sequence of the repetitive unit. When repetitive units are in a head-to-head orientation, one repetitive unit has a sequence that corresponds to the ordinary N-terminus to C-terminus amino acid sequence of the repetitive unit, while the other repetitive unit has a sequence that is reversed in comparison to the ordinary N-terminus to C-terminus amino acid sequence of the repetitive unit. That is, the reversed repetitive unit has a sequence that corresponds to the ordinary polynucleotide or amino acid sequence when such sequences are read in the C-terminus to N-terminus direction (polypeptide) or 3′-5′ direction (polynucleotide encoding a repetitive unit). The silk polypeptides can contain an intervening amino acid sequence between the repetitive units when the repetitive units are linked either in a head-to-tail or head-to-head orientation.

“Repetitive unit,” as used herein, refers to a silk polypeptide monomer or a portion thereof which corresponds in amino acid sequence to a region of iterated peptide motifs within a naturally-occurring silk polypeptide (e.g., MaSpI, ADF-3, or Flag) found in an spider or insect biofilament, or to a sequence substantially similar to such a sequence. The “repetitive unit” does not include the non-repetitive hydrophilic amino acid domain generally thought to be present at the carboxyl terminus of naturally-occurring silk monomers, as described herein. At a minimum, a “repetitive unit” comprises a combination of the iterated peptide motifs known by those of skill in the art to be present within a particular naturally-occurring silk monomer. For example, a “repetitive unit” can be a portion of a polypeptide corresponding to all or part of the repetitive regions of MaSpI, MaSpII, and/or ADF-3, e.g., SEQ ID NOS:1, 2 and/or 3, that are shown in FIGS. 5, 6 and 7, respectively; or any of the consensus motifs or repeat units ascribed to spider or lepidopteran silks, or synthetic polymeric units described in general formulae that when polymerized are intended to mimic spider or lepidopteran silk properties, as are described in U.S. Pat. Nos. 6,268,169, 6,184,348, 6,018,030, 5,994,099, 5,989,894, 5,514,581, 5,728,810, 5,756,677, 5,733,771, each incorporated by reference herein in its entirety. Further by example, a “repetitive unit” can comprise peptide sequences such as those identified as SEQ ID NOS:4-27 that are motifs common to silks. A “repetitive unit” need not contain a sequence corresponding to every single iterated peptide motif present within a particular naturally-occurring silk monomer. However, the repetitive unit is formulated to confer on a biofilament composed of isolated silk polypeptides properties of, e.g. strength and/or elasticity similar to those associated with naturally-occurring silk.

“Substantially identical,” as used herein, refers to a polypeptide or nucleic acid exhibiting at least about 50%, about 70%, about 85%, about 90%, about 95%, or even about 99% identity to a reference amino acid or nucleic acid sequence. Unless otherwise specified for polypeptides, the length of comparison of sequences will generally be at least 20 amino acids, preferably at least 30 amino acids, more preferably at least 40 amino acids, and most preferably at least 50 amino acids. Unless otherwise specified for nucleic acids, the length of comparison sequences will generally be at least 60 nucleotides, preferably at least 90 nucleotides, and more preferably at least 120 nucleotides.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used. Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The term “about,” as used herein, unless otherwise indicated, refers to a value that is no more than 10% above or below the value being modified by the term.

In the event the modified value must be an integer, the resulting modified value will be an integer that is no more than 10% above or below the original value. Further, in instances wherein 10% of the value being modified by this term results in a value less than one, then it is understood that, as used herein, that the modified value is 1; in the event that the upper limit of the modified value is less than one integer greater than the value being modified, the modified value is understood to be an integer that is 1 greater than the original value.

### 5. DESCRIPTION OF DRAWINGS

FIG. 1 is a schematic showing DNA expression constructs used to produce recombinant (rc)-dragline spider silk polypeptides in mammalian cells.

FIGS. 2A and 2B are photographs showing the detection, by Western blot analysis, of ADF-3 and MaSpII (FIG. 2A), and MaSpI (FIG. 2B) spider silk proteins secreted into the media from BHK cells. Approximately 20 μl of conditioned media was loaded per lane. FIG. 2A: Lane 1: ADF-3 His; Lane 2: ADF-3; Lane 3: ADF-33; Lane 4: ADF-333; and Lane 5: MaSpII. FIG. 2B: Lane 1: MaSpI; and Lane 2: MaSpI(2).

FIGS. 3A and 3B are photographs of a silver stained SDS-PAGE gel and a Western blot analysis, respectively, showing the purification of ADF-3 rc-spider silk polypeptide secreted from mammalian cells. FIG. 3A: Lane 1: molecular weight markers (kDa); Lane 2: solubilized proteins following ammonium sulfate precipitation of BHK conditioned media loaded onto an anion exchange column; Lane 3: flow through protein fraction from anion exchange column; Lane 4: elution fraction of bound proteins from anion exchange column. FIG. 3B: Lanes 1-3: same as lanes 2-4 in FIG. 3A.

FIG. 4 depicts exemplary structures of multimeric constructs encompassed by the present invention.

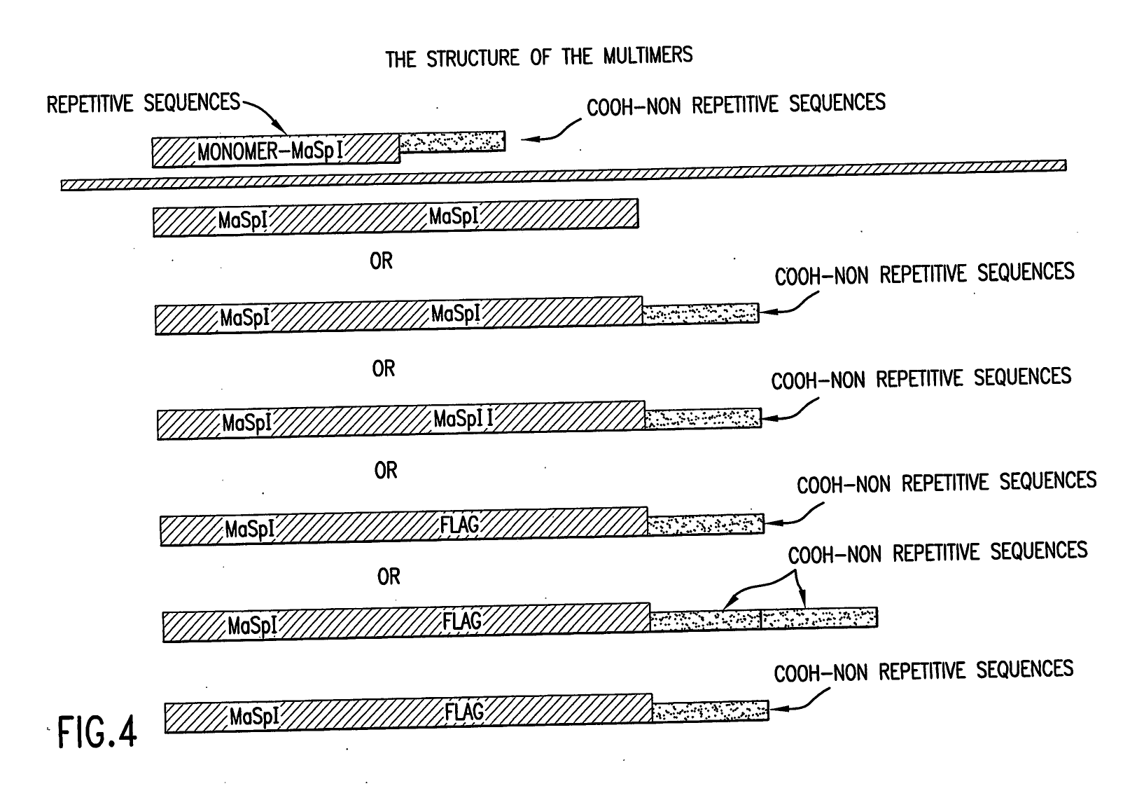

FIG. 5 depicts the amino acid sequence of a representative MaSpI silk polypeptide which may be recovered according to the methods of the invention, arranged so that the amino acid repeat motifs can be observed.

FIG. 6 depicts the amino acid sequence of a representative MaSpII silk polypeptide which may be recovered according to the methods of the invention, arranged so that the amino acid repeat motifs can be observed.

FIG. 7 depicts the amino sequence of a representative ADF-3 polypeptide which may be recovered according to the methods of the invention, arranged so that the amino acid repeat motifs can be observed.

FIG. 8 depicts the amino sequence of a representative ADF-1 polypeptide which may be recovered according to the methods of the invention.

FIG. 9 depicts the amino sequence of a representative ADF-2 polypeptide which may be recovered according to the methods of the invention.

FIG. 10 depicts the amino sequence of a representative ADF-4 polypeptide which may be recovered according to the methods of the invention.

### 6. DETAILED DESCRIPTION OF THE INVENTION

The present invention relates to silk polypeptides, methods of expressing and purifying such silk polypeptides, and methods of spinning such silk polypeptides into biofilaments having useful physical properties, e.g., strength and elasticity. In certain aspects, the invention provides isolated silk polypeptides comprised of a plurality of repetitive units and a non-repetitive hydrophilic amino acid domain, wherein the isolated silk polypeptide has a molecular weight ranging from about 16 kDa to about 800 kDa. In other aspects, the invention provides polynucleotides encoding the silk polypeptides described herein, vectors comprising such polynucleotides, and cells, plants and mammals transformed with vectors comprising such polynucleotides.

In yet other aspects, the invention provides methods of producing the silk polypeptides of the invention comprising culturing in cell culture media a host cell containing a nucleic acid encoding a silk polypeptide of the invention under conditions that cause the host cell to express the silk polypeptide and purifying the silk polypeptide from the host cell or from the cell culture media. In other aspects, the invention also provides methods of producing the silk polypeptides of the invention comprising generating a transgenic non-human animal that expresses a nucleic acid molecule encoding the silk polypeptide of the invention and recovering the silk polypeptide from a biological fluid produced by the transgenic animal. In still other aspects, the invention provides methods of producing a biofilament composed of a plurality of isolated silk polypeptides, comprising expressing a silk polypeptides of the invention in a transformed or transfected host cell or in a biological fluid of a transgenic ruminant, purifying a plurality of the silk polypeptides, and spinning the purified plurality of silk polypeptides to form a biofilament. In certain aspects, the invention provides biofilaments produced according to the methods of the invention as well as biofilaments comprised of a plurality of the isolated silk polypeptides of the invention.

These isolated silk polypeptides, polynucleotides encoding such polypeptides, and methods of producing and using silk polypeptides are based in part on Applicants' discovery that inclusion of a non-repetitive hydrophilic domain in a silk polypeptide gives desirable physical characteristics and/or functionality. While not intending to bound by any particular theory or mechanism of action, the non-repetitive hydrophilic amino acid domain is believed to increase the solubility of the silk polypeptides and/or aid the trafficking and/or secretion of silk polypeptides when expressed in host cells, allowing for the expression of larger silk polypeptides than was previously possible. These larger silk polypeptides are useful for forming biofilaments with desirable physical characteristics, e.g., strength and elasticity.

### 6.1 Silk Polypeptides

In certain aspects, the invention provides isolated silk polypeptides comprising a plurality of repetitive units and a non-repetitive hydrophilic amino acid domain, wherein the isolated silk polypeptide has a molecular weight ranging from about 16 kDa to about 800 kDa. Repetitive units and non-repetitive hydrophilic amino acid domains are described in Sections 6.1.1 and 6.1.2, respectively, below. In certain embodiments, the isolated silk polypeptide can have a molecular weight ranging from about 58 kDa to about 800 kDa. In other embodiments, the isolated silk polypeptide can have a molecular weight of about 65 kDa to about 800 kDa. In yet other embodiments, the isolated silk polypeptide can have a molecular weight ranging from about 70 kDa to about 800 kDa. In still other embodiments, the isolated silk polypeptide can have a molecular weight ranging from about 100 kDa to about 800 kDa. In still other embodiments, the isolated silk polypeptide can have a molecular weight ranging from about 150 kDa to about 800 kDa In yet other embodiments, the isolated silk polypeptide can have a molecular weight ranging from about 200 kDa to about 800 kDa. In still other embodiments, the isolated silk polypeptide can have a molecular weight ranging from about 250 kDa to about 800 kDa. In yet other embodiments, the isolated silk polypeptide can have a molecular weight ranging from about 300 kDa, about 350 kDa, about 400 kDa, about 450 kDa, about 500 kDa, about 550 kDa, about 600 kDa, about 650 kDa, about 700 kDa, about 750 kDa, to about 800 kDa. In still other embodiments, the isolated silk polypeptide can have a molecular weight ranging from about 500 kDa to about 800 kDa.

In certain embodiments, the isolated silk polypeptide can have a molecular weight ranging from about 16 kDa to about 60 kDa. In other certain embodiments, the isolated silk polypeptide can have a molecular weight ranging from about 16 kDa to about 100 kDa. In other embodiments, the isolated silk polypeptide can have a molecular weight ranging from about 100 kDa to about 300 kDa. In other embodiments, the isolated silk polypeptide can have a molecular weight ranging from about 55 kDa to about 100 kDa. In other embodiments, the silk polypeptide have a molecular weight range at least about 58 kDa to about 210 kDa. In still other embodiments, the isolated silk polypeptide can have a molecular weight ranging from about 70 kDa to about 140 kDa. In yet other embodiments, the isolated silk polypeptide can have a molecular weight ranging from about 100 kDa to about 150 kDa. In yet other embodiments, the isolated silk polypeptide can have a molecular weight ranging from about 150 kDa to about 200 kDa. In still other embodiments, the isolated silk polypeptide can have a molecular weight ranging from about 200 kDa to about 250 kDa. In yet other embodiments, the isolated silk polypeptide can have a molecular weight ranging from about 250 kDa to about 300 kDa. In still other embodiments, the isolated silk polypeptide can have a molecular weight ranging from about 300 kDa to about 500 kDa. In still other embodiments, the isolated silk polypeptide can have a molecular weight ranging from about 65 kDa, about 70 kDa, about 75 Da, about 80 kDa, about 85 kDa, about 90 kDa, about 95 kDa, about 100 kDa, about 150 kDa, about 200 kDa, about 250 kDa, about 300 kDa, or about 350 kDa, to about 400 kDa, about 450 Da, about 500 kDa, about 550 kDa, about 600 kDa, about 650 kDa, about 700 kDa, about 750 kDa, or about 800 kDa.

The silk polypeptides of the invention may be monomeric proteins, fragments thereof, or dimers, trimers, tetramers, or other multimers of a monomeric protein.

### 6.1.1. Repetitive Units of Silk Polypeptides

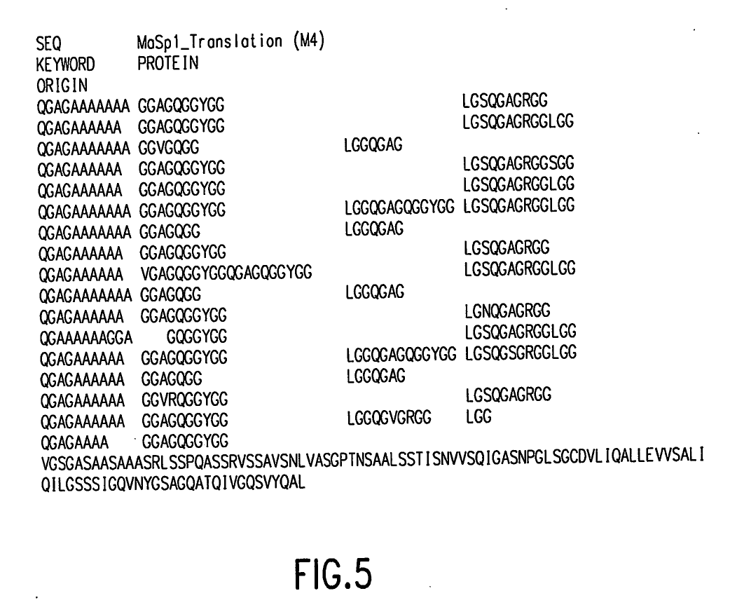

A repetitive unit of a silk polypeptide, as defined above, refers to a silk polypeptide monomer or a portion thereof which corresponds in amino acid sequence to a region of iterated peptide motifs within a naturally-occurring silk polypeptide (e.g., MaSpI, ADF-3, or Flag) found in an spider or insect biofilament, or to a sequence substantially similar to such a sequence. When made in reference to polynucleotide, a repetitive unit is that portion of the polynucleotide encoding a repetitive unit as defined above. In a preferred embodiment, the amino acid sequence of each repetitive unit is selected from the group consisting of the amino acid sequences of SEQ ID NOS:1-3, as shown in FIGS. 5, 6 and 7, respectively. In other embodiments, a repetitive unit can be a portion of a polypeptide corresponding to any of the consensus motifs or repeat units ascribed to spider or lepidopteran silks, or synthetic polymeric units described in general formulae that when polymerized are intended to mimic spider or lepidopteran silk properties, as are described in U.S. Pat. Nos. 6,268,169, 6,184,348, 6,018,030, 5,994,099, 5,989,894, 5,514,581, 5,728,810, 5,756,677, 5,733,771, each incorporated by reference herein in its entirety. In still other embodiments, the repetitive units comprise repetitive units forming an amorphous domain and a crystal-forming domains. Preferably, such repetitive units comprise amino acid sequences identified as SEQ ID NO:28 and SEQ ID NO:29.

The silk polypeptide repetitive units according to the present invention can be derived from any the repetitive regions of any silk polypeptide known to one of skill in the art without limitation, including polypeptides derived from spider silks such as major ampullate, minor ampullate, flagelliform, tubuliform, aggregate, aciniform, and pyriform silks as well as polypeptides derived from insect silks. The silk polypeptides can be from any type of silk-producing spider or insect, including, but not limited to, those produced by Nephila clavipes, Araneus ssp., (including A. diadematus and A. bicentenarius) and from the order Lepidoptera (for example, Bombyx mori). Dragline silk produced by the major ampullate gland of Nephilia clavipes occurs naturally as a mixture of at least two proteins, designated as MaSpI and MaSpII. Similarly, dragline silk produced by A. diadematus is also composed of a mixture of two proteins, designated ADF-3 and ADF-4.

Spider silk polypeptides are dominated by iterations of four amino acid motifs: (1) polyalanine (Aₙ); (2) alternating glycine and alanine (GA)ₙ; (3) GGX; and (4) GPG(X)ₙ, where X represents a small subset of amino acids, including A, Y, L and Q (for example, in the case of the GPGXX motif, GPGQQ is the major form). Hayashi et al., J. Mol. Biol. 275:773 (1998); Hinman et al., Trends in Biotech 18:374-379 (2000). As such, the repetitive units of the silk polypeptides of the invention can comprise iterated peptide motifs such as these.

Spider silk proteins may also contain spacers or linker regions comprising charged groups or other motifs, which separate the iterated peptide motifs into clusters or modules. As such the silk polypeptides of the invention can also comprise such spacers or linker regions.

Modules of the GPG(X)ₙ motif form a β-turn spiral structure which imparts elasticity to the protein. Major ampullate and flagelliform silks both have a GPGXX motif and are the only silks which have elasticity greater than 5-10%. Major ampullate silk, which has an elasticity of about 35%, contains an average of about five β-turns in a row, while flagelliform silk, which has an elasticity of greater than 200%, has this same module repeated about 50 times. The polyalanine (Aₙ) and (GA)ₙ motifs form a crystalline β sheet structure that provides strength to the proteins. The major ampullate and minor ampullate silks are both very strong, and at least one protein in each of these silks contains a (Aₙ)/(GA)ₙ module. The GGX motif is associated with a helical structure having three amino acids per turn (3₁₀ helix), and is found in most spider silks. The GGX motif may provide additional elastic properties to the silk. Accordingly, in certain embodiments, repetitive units are such amino acid sequences, e.g., ones encompassed by the generalized formulae of the motifs Aₙ, GAₙ, GGX, GPG(X)ₙ, where X represents the amino acid A, Q, G, L, S, Y or V, and n represents an integer from 1 to about 8. In other embodiments, the invention provides isolated silk polypeptides comprising a plurality of repetitive units and a non-repetitive hydrophilic amino acid domain, wherein the repetitive units comprise amino acid sequences that form secondary structures selected from the group consisting of: β-turn spiral, crystalline β sheet, and 3₁₀ helix.

Methods and composition of the present invention are applicable to silk polypeptides which comprise the above-mentioned motifs. In particular, the silk polypeptids of the invention can comprise a non-repetitive hydrophillic amino acid domain and a plurality of repetitive units that have a sequence that is substantially identical or identical to a sequence selected from a plurality or combination of the group consisting of:

| | |
|---|---|
| AAAAA | (SEQ ID NO: 4) |
| GAGA | (SEQ ID NO: 5) |
| GAGAGA | (SEQ ID NO: 6) |
| GAGAGAGA | (SEQ ID NO: 7) |
| GAGAGAGAGA | (SEQ ID NO: 8) |
| GAGAGAGAGAGA | (SEQ ID NO: 9) |
| GAGAGAGAGAGAGA | (SEQ ID NO: 10) |
| GGYGQGY | (SEQ ID NO: 11) |
| AAAAAAAA | (SEQ ID NO: 12) |
| GGAGQGGY | (SEQ ID NO: 13) |
| GGQGGQGGYGGLGSQGA | (SEQ ID NO: 14) |
| ASAAAAAA | (SEQ ID NO: 15) |
| GPGQQ | (SEQ ID NO: 16) |
| (GPGQQ)₂ | (SEQ ID NO: 17) |
| (GPGQQ)₃ | (SEQ ID NO: 18) |
| (GPGQQ)₄ | (SEQ ID NO: 19) |
| (GPGQQ)₅ | (SEQ ID NO: 20) |
| (GPGQQ)₆ | (SEQ ID NO: 21) |
| (GPGQQ)₇ | (SEQ ID NO: 22) |
| (GPGQQ)₈ | (SEQ ID NO: 23) |
| GPGGQGGPYGPG | (SEQ ID NO: 24) |
| SSAAAAAAAA | (SEQ ID NO: 25) |
| GPGSQGPS | (SEQ ID NO: 26) |
| and | |
| GPGGY. | (SEQ ID NO: 27) |

Further, the methods of the present invention encompass spinning biofilaments from silk polypeptides such as those discussed above.

Preferably, the silk polypeptide has a repetitive unit creating both an amorphous domain and a crystal-forming domain, particularly one having a sequence that is identical to or substantially identical to: AGQGGYGGLGSQGAGRGGLGGQGAGAAAAAAAGG (SEQ ID NO:28), of Nephila spidroin 1 MaSpI) proteins. In another embodiment, it is preferred that the silk polypeptide has a consensus structure that is identical to or substantially identical to: CPGGYGPGQQCPGGYGPGQQCPGGYGPGQQGPSGPGSAA AAAAAAAA (SEQ ID NO:29), of Nephila spidroin 2 (MaSpII) proteins. Preferably, the silk polypeptides when subjected to shear forces and mechanical extension, for example in forming a biofilament, has a polyalanine segment that undergoes a helix to a β-sheet transition, where the transition forms a β-sheet that stabilizes the structure of the protein. It is also preferred that the protein has an amorphous domain that forms a β-pleated sheet such the inter-β sheet spacings are between about 3 and about 8 angstroms; preferably between about 3.5 and about 7.5 angstroms.

The sequences of the spider silk polypeptides, disclosed herein, may have additional amino acids or amino acid sequences inserted into the polypeptide, in the middle thereof, or at the ends thereof, so long as the protein possesses substantial similarity to the amino acid sequences of the repetitive units described herein and/or the polypeptides can be spun into biofilaments when having desired physical characteristics. Likewise, some of the amino acids or amino acid sequences may be deleted from the polypeptide so long as the polypeptide substantial similarity to the amino acid sequences of the repetitive units described herein and/or the polypeptides can be spun into biofilaments when having desired physical characteristics. Amino acid substitutions may also be made in the sequences so long as the polypeptide substantial similarity to the amino acid sequences of the repetitive units described herein and/or the polypeptides can be spun into biofilaments when having desired physical characteristics. For example, a biofilament desirably exhibits a toughness of at least 0.6 gpd and a tenacity of at least about 1.7 gpd.

In other aspects, the invention provides isolated silk polypeptides comprising a plurality of repetitive units and a non-repetitive hydrophilic amino acid domain, wherein a at least one of the repetitive units can have an amino acid sequence that is in a reversed order in comparison to the naturally-occurring amino terminus to carboxyl terminus amino acid sequence. For example, one of the repetitive units can have an amino acid sequence that is the amino acid sequence of MaSpI as presented in FIG. 5, except that the sequence is read from the carboxyl end of the repetitive unit to the amino end of the repetitive unit, rather than the conventional amino-terminal end to carboxyl-terminal end, or the iterated peptide motifs may comprise (AG)ₙ rather than (GA)ₙ.

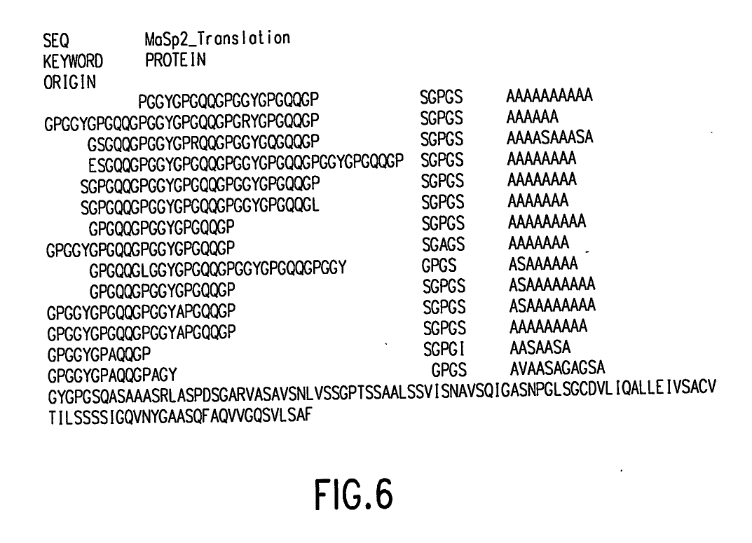

Examples of recombinantly produced MaSpI and MaSpII silk polypeptides that may be used as part of the silk polypeptides of the invention are depicted in FIGS. 5 and 6, respectively. FIG. 5 shows the sequence of a representative MaSpI protein arranged so that the amino acid repeat motifs can be seen. FIG. 6 shows the sequence of a representative MaSpII protein, arranged so that the amino acid repeat motifs can be seen.

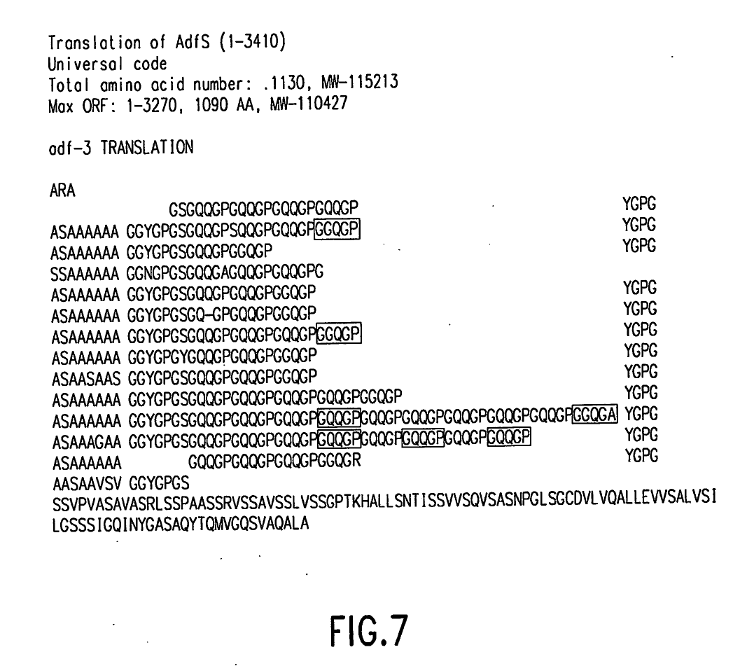

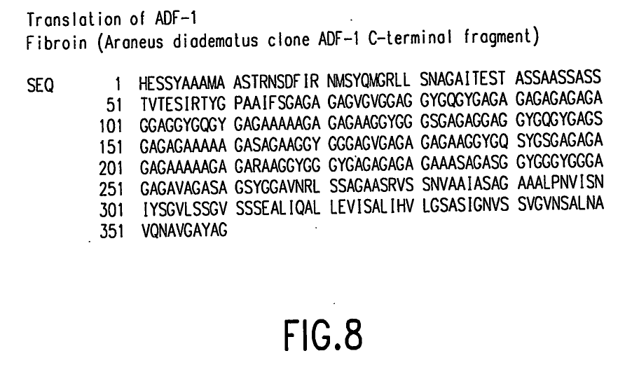

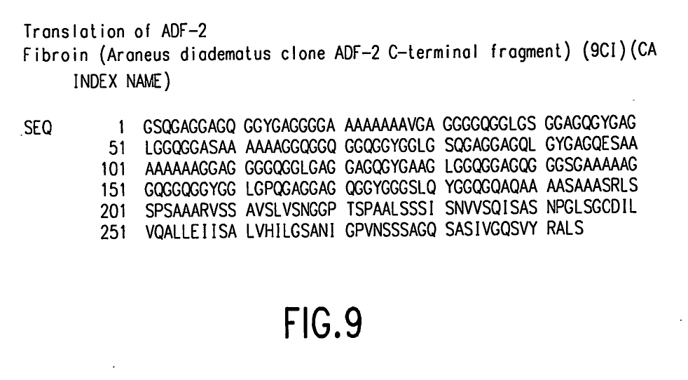

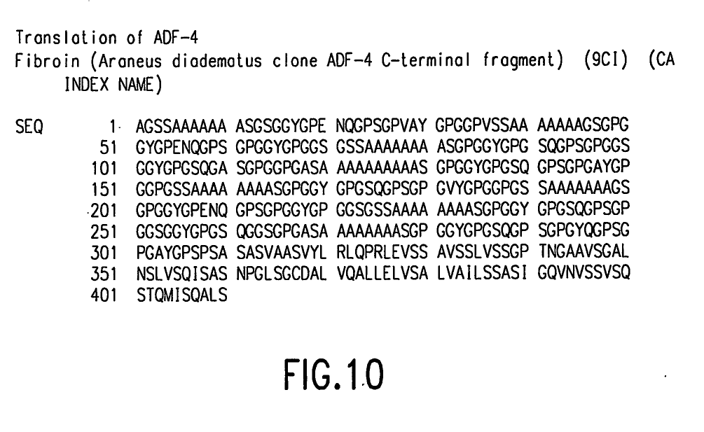

Recombinantly produced ADF-1, ADF-2, ADF-3 and ADF-4 silk polypeptide repetitive regions may also be used in the present invention. These proteins are produced naturally by the Araneus diadematus species of spider. The ADF-1 repetitive region generally comprises 68% poly(A)₅ or (GA)₂₋₇, and 32% GGYGQGY. The ADF-2 repetitive region generally comprises 19% poly(A)₈, and 81% GGAGQGGY and GGQGGQGGYGGLGSQGA. The ADF-3 repetitive region generally comprises 21% ASAAAAAA and 79% (GPGQQ)ₙ, where n=1-8. The ADF-4 repetitive region comprises 27% SSAAAAAA and 73% GPGSQGPS and GPGGY. An example of a recombinantly produced ADF-3 protein which may be used in the invention is depicted in FIG. 7, which shows the sequence of a representative ADF-3 protein, arranged so that the amino acid repeat motifs in the repetitive region can be seen. The amino acid sequences of ADF-1, ADF-2, and ADF-4 are presented in FIGS. 8, 9, and 10, respectively.

Abbreviations for amino acids used herein are conventionally defined as described herein below unless otherwise indicated.

| | One-Letter | Three Letter |
|---|---|---|
| Amino Acid | Abbreviation | Abbreviation |
| Alanine | A | Ala |
| Arginine | R | Arg |
| Asparagine | N | Asn |
| Aspartic acid | D | Asp |
| Asparagine or aspartic acid | B | Asx |
| Cysteine | C | Cys |
| Glutamine | Q | Gln |
| Glutamic acid | E | Glu |
| Glutamine or glutamic acid | Z | Glx |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

### 6.1.2. Non-Repetitive Hydrophilic Domains

The invention provides isolated silk polypeptides comprised of a plurality of repetitive units and a non-repetitive hydrophilic amino acid domain. The term “non-repetitive is not meant to connote that the amino acid sequence of the non-repetitive hydrophilic amino acid domain does not contain any repeated sequences; rather, the term “non-repetitive” distinguishes the non-repetitive amino acid domain from the highly repetitive repetitive units. Thus, the non-repetitive hydrophilic amino acid domain can contain some repetitive sequences but are not composed of the iterated peptide motifs found in the repetitive units.

In certain embodiments, the non-repetitive hydrophilic amino acid domain can be toward the carboxyl terminus with respect to the repetitive units. That is, the hydrophilic amino acid domain is present on the silk polypeptide at a position carboxyl to the most carboxyl repetitive unit. In one such embodiment, the hydrophilic amino acid is at the carboxyl terminus of the silk polypeptide. In other embodiments, the non-repetitive hydrophilic amino acid domain can be toward the amino terminus with respect to the repetitive units. That is, the hydrophilic amino acid domain is present on the silk polypeptide at a position amino to the most carboxyl repetitive unit. In one such embodiment, the hydrophilic amino acid is at the amino terminus of the silk polypeptide. In yet other embodiments, the non-repetitive hydrophilic amino acid domain can be between two of the repetitive units. In other aspects, the invention further provides isolated silk polypeptides having a plurality of repetitive units and a non-repetitive hydrophilic amino acid domain, further comprising a proteolytic site, wherein cleavage at the proteolytic site cleaves the non-repetitive hydrophilic amino acid domain from a repetitive unit. In other embodiments, the invention further provides isolated silk polypeptides having a plurality of repetitive units and a non-repetitive hydrophilic amino acid domain, further comprising a first proteolytic site and a second proteolytic site, wherein cleavage at the first proteolytic site and at the second proteolytic site cleaves the non-repetitive hydrophilic amino acid domain from the repetitive units.

The most highly conserved coding sequences between Nephila silk polypeptides lies in the last 97 amino acids (Beckwitt & Arcidiacono, J. Biol. Chem. 269:6661-6663 (1994)). The carboxyl terminal domain of all spider polypeptides cloned to date show strong identity, and they contain a highly conserved cysteine residue. While not intending to be bound by any particular theory or mechanism of action, the non-repetitive hydrophilic amino acid domain may increase the solubility of the silk polypeptides as compared to polypeptides that are only repetitive units, or as encoded in polynucleotides, result in the stabilization of mRNA encoding silk polypeptides. An alternative theory is that the non-repetitive hydrophilic amino acid domain assists in trafficking and/or secretion of the silk polypeptides. Accordingly, the non-repetitive hydrophilic amino acid domain can be any non-repetitive hydrophilic amino acid domain known by one of skill in the art to increase the solubility of a silk polypeptide relative to a silk polypeptide without a non-repetitive hydrophilic amino acid domain and/or assist in trafficking and/or secretion of a silk polypeptide, without limitation.

In certain embodiments, the non-repetitive hydrophilic amino acid domain can be a polypeptide comprising about 25 to about 150 amino acids, at least about 20% of which are hydrophilic amino acids. In other embodiments, the non-repetitive hydrophilic amino acid domain can be a polypeptide comprising about 25 to about 150 amino acids, at least about 30% of which are hydrophilic amino acids. In still other embodiments, the non-repetitive hydrophilic amino acid domain can be a polypeptide comprising about 25 to about 150 amino acids, at least about 40% of which are hydrophilic amino acids. In yet other embodiments, the non-repetitive hydrophilic amino acid domain can be a polypeptide comprising about 25 to about 150 amino acids, at least about 50% of which are hydrophilic amino acids. In still other embodiments, the non-repetitive hydrophilic amino acid domain can be a polypeptide comprising about 25 to about 150 amino acids, at least about 60% of which are hydrophilic amino acids. In yet other embodiments, the non-repetitive hydrophilic amino acid domain can be a polypeptide comprising about 25 to about 125 amino acids, at least about 60% of which are hydrophilic amino acids. A hydrophilic amino acid is one that exhibits a hydrophobicity of less than zero according to the normalized consensus hydrophobicity scale of Eisenberg et al., J. Mol. Biol. 179:125-142 (1984), and include Thr (T), Ser (S), H is (H), Glu (E), Asn (N), Gln (O), Asp (D), Lys K) and Arg (R).

In certain embodiments, the non-repetitive hydrophilic amino acid domain can have an amino acid sequence that is identical or substantially identical to sequences selected from the group consisting of amino acid sequences of non-repetitive hydrophilic carboxyl terminal regions of MaSpI, MaSpII, MiSpI, MiSpII, ABF-1, ADF-1, ADF-2, ADF-3, ADF-4, NCF-1, NCF-2, and Flag. The sequences of the non-repetitive hydrophilic carboxyl terminal regions of ADF-1, ADF-2, ADF-4, and ABF-1 may be found in Guerette et al., 1996, Science 272:(112-115), hereby incorporated by reference in its entirety, while the amino acid sequences of the non-repetitive hydrophilic carboxyl terminal regions of MaSpI, MaSpI, and ADF-3 are presented in FIGS. 5-7, respectively. The sequences of the non-repetitive hydrophilic carboxyl terminal regions of MiSpI and MiSpII may be found in U.S. Pat. No. 5,756,677, which is hereby incorporated by reference in its entirety. The non-repetitive hydrophilic carboxyl terminal sequences of flagelliform (Flag) and the Araneus bicentenarius silk protein ABF-1 may be found in U.S. Pat. No. 5,995,099 and Beckwitt & Arcidiacono, J. Biol. Chem. 269:6661-6663 (1994), both hereby incorporated by reference in their entirety. In other embodiments, the non-repetitive hydrophilic amino acid domain can comprise a consensus sequence derived from the non-repetitive carboxyl termini regions of major ampullate and ADF-1, ADF-2, ADF-3, and ADF-4 sequences.

In certain preferred embodiments, the non-repetitive hydrophilic amino acid domain can have an amino acid sequence that is selected from the group consisting of the 109 amino acids found at the carboxyl terminus of MaSpI, the 109 amino acids found at the carboxyl terminus of MaSpII, and the 108 amino acids found at the carboxyl terminus of ADF-3, each as shown in FIGS. 5, 6 and 7, respectively.

In certain embodiments, the non-repetitive hydrophilic domain can have a cysteine residue present, which can be used, for example, to allow dimer formation between polypeptide subunits.

In other aspects, the invention provides isolated silk polypeptides having a plurality of repetitive units and a non-repetitive hydrophilic amino acid domain, wherein the silk polypeptide can be precipitated and subsequently redissolved in an aqueous buffer. An aqueous buffer can include any water-based solution known to one of skill in the art without limitation. In a preferred embodiment, the aqueous buffer is 20 mM glycine at pH 10. In another embodiment, the aqueous buffer is standard phosphate-buffered saline.

In yet other aspects, the invention provides isolated silk polypeptides having a plurality of repetitive units and a non-repetitive hydrophilic amino acid domain, further comprising one or more additional non-repetitive hydrophilic amino acid domains. In certain embodiments, the one or more additional non-repetitive hydrophilic amino acid domains comprises at least about 2 to about 4 non-repetitive hydrophilic amino acid domains.

### 6.1.3. Optional Features of Silk Polypeptides

In certain aspects, the invention also provides isolated silk polypeptides which comprise additional optional features. In certain embodiments, the isolated silk polypeptides further comprise a proteolytic site, wherein cleavage at the proteolytic site results in the separation of all, substantially all, or a portion of the non-repetitive hydrophilic amino acid domain from a repetitive unit. In certain embodiments, the isolated silk polypeptides further comprise a proteolytic site, wherein cleavage at the proteolytic site results in the separation of all, substantially all, or a portion of the non-repetitive hydrophilic amino acid domain from the repetitive units. In other embodiments, the isolated silk polypeptides further comprise a first proteolytic site and a second proteolytic site, wherein cleavage at the first proteolytic site and at the second proteolytic site cleaves all, substantially all, or a portion of the non-repetitive hydrophilic amino acid domain from the repetitive units. In still other embodiments the non-repetitive hydrophilic domain can contain a proteolytic site that can be located such that cleavage at the proteolytic site can remove the non-repetitive hydrophilic amino acid domain from the non-repetitive units.

In certain embodiments, all, substantially all, or a portion of the non-repetitive hydrophilic amino acid domain can be cleaved from the repetitive units endogenously within the expression system before purification of the silk polypeptides. In further embodiments, all, substantially all, or a portion of the non-repetitive hydrophilic amino acid domain can be cleaved from the repetitive units before, during, or after secretion of the silk polypeptides into a biological fluid, including milk of a lactating female mammal or urine, before purification of the silk polypeptides. In other embodiments, all, substantially all, or a portion of the non-repetitive hydrophilic amino acid domain can be cleaved from the repetitive units following purification of the silk polypeptides.

The proteolytic site can be any proteolytic site known to one of skill in the art without limitation. In certain embodiments, the proteolytic site can be subject to cleavage by a protease. In other embodiments, the proteolytic site can be subject to cleavage by chemical treatment.

In embodiments where the proteolytic site is subject to cleavage with a protease, the proteolytic site can be a proteolytic site that is recognized and cleaved by any protease known by one of skill in the art without limitation. In certain embodiments, the proteolytic site can be a proteolytic site that is recognized and cleaved by a serine protease, e.g., chymotrypsin, trypsin, elastase, subtilisin, etc.; a cysteine (thiol) protease, e.g., bromelain, papain, cathepsins, etc.; an aspartic protease; e.g., pepsin, cathepsins, renin, etc.; and a metallo-protease, e.g., thermolysin, collagenase, etc. In certain embodiments, the proteolytic site can be a proteolytic site that is recognized by Arg-C proteinase, Asp-N endopeptidase, or Glutamyl endopeptidase. In a preferred embodiment, the proteolytic site is a proteolytic site that is recognized and cleaved by trypsin.

In embodiments where the proteolytic site is subject to cleavage by chemical treatment, the proteolytic site can be a proteolytic site that is recognized and cleaved by any chemical treatment known by one of skill in the art without limitation. In certain embodiments, the proteolytic site can be a proteolytic site that is recognized and cleaved by a chemical treatment selected from the group of cyanogen bromide, BNPS-skatole (2-(2-nitrophenylsulfenyl)-3-methylindole), o-lodosobenzoic acid, Cyssor ((2-methyl) N-1-benzenesulfonyl-N-4-(bromoacetyl)quinone diimide), NTCB (2-nitro-5-thiocyanobenzoic acid), and hydroxylamine.

In other aspects, the isolated silk polypeptides of the invention can optionally further comprise a secretory signal peptide sequence. The secretory signal peptide sequence can be any secretory signal peptide sequence known by one of skill in the art without limitation. In certain embodiments, the secretory signal peptide sequence can be a secretory signal peptide sequence that directs secretion of a polypeptide from a prokaryotic cell. In other embodiments, the secretory signal peptide sequence can be a secretory signal peptide sequence that directs secretion of a polypeptide from a eukaryotic cell. In other embodiments, the secretory signal peptide can a secretory signal peptide sequence that directs translocation of a polypeptide in plants. In further embodiments, the secretory signal peptide sequence can be a secretory signal peptide sequence that directs secretion of a polypeptide from a eukaryotic cell of a non-human mammal. In still further embodiments, the secretory signal peptide sequence can be a secretory signal peptide sequence that directs secretion of a polypeptide from a cell of a particular tissue of a non-human mammal. In certain embodiments, the secretory signal sequence can be derived from the same gene as the promoter used to drive expression of the silk polypeptides of the invention. For example, the secretory signal sequence can be derived from the genes which encode whey acidic protein, αSS1-casein, αS2-casein, β-casein, κ-casein, β-lactoglobin, α-lactalbumin, uroplakin, uromodulin or rennin. In a preferred embodiment, the secretory signal sequence is an Ig-kappa secretory signal sequence.

In other aspects, the isolated silk polypeptides of the invention can optionally further comprise a tag that assists in purification of the silk polypeptides or identification of the silk polypeptides in extracts. The tag that assists in purification of the silk polypeptide can be any tag useful for such purposes that is known to one of skill in the art without limitation. In certain embodiments, the label can be a c-myc epitope. In other embodiments, the label can be a histidine tag.

### 6.2. Polynucleotides Encoding Silk Polypeptides

The silk polypeptides are encoded by nucleic acids, which can be joined to a variety of expression control elements, including microbial, plant, or tissue-specific animal promoters, enhancers, secretory signal sequences, and terminators. These expression control sequences, in addition to being adaptable to the expression of a variety of gene products, afford a level of control over the timing and extent of production.

### 6.3. Silk Polypeptide Vectors

Also included in the invention are those promoter elements which are sufficient to render promoter-dependent gene expression controllable for cell type-specific, tissue-specific and developmental stage specific (e.g., lactation) expression of silk polypeptides. Such elements may be located in the 5′ or 3′ regions or both of the encoded polypeptide. Desired promoters of the invention direct transcription of a protein in a milk-producing cell; such promoters include, without limitation, promoters from the following genes: whey acidic protein, αS1-casein, αS2-casein, β-casein, κ-casein, β-lactoglobin, and α-lactalbumin. Other useful promoters of the invention direct transcription of a protein in a urine-producing cell (e.g., a uroepithelial cell or a kidney cell); such promoters include, without limitation, the promoter from the uroplakin, uromodulin or rennin genes. Yet another desired promoter of the invention directs transcription of a protein in an embryonal cell.

### 6.4. Recombinant Sources of Silk Polypeptides

The silk polypeptides of the invention may be produced by expressing the proteins in cell culture, in transgenic animals, and in transgenic plants. Each of these expression systems is described below.

### 6.4.1 Silk Polypeptides from Cell Culture

The silk polypeptides of the invention can be produced by any method known in the art for the protein synthesis, in particular, by recombinant expression techniques.

The nucleotide sequence encoding a silk polypeptide repetitive unit may be obtained from any information available to those of skill in the art (i.e., from Genbank, the literature, or by routine cloning) coupled with the teaching provided herein. If a clone containing a nucleic acid encoding a polypeptide sequence is not available, but the sequence of the polypeptide itself is known, a nucleic acid encoding the immunoglobulin may be chemically synthesized or obtained from a suitable source (e.g., a cDNA library, or a cDNA library generated from, or nucleic acid, preferably poly A⁺ RNA, isolated from any tissue or cells expressing the polypeptide) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the polypeptide. Amplified nucleic acids generated by PCR may then be cloned into replicable cloning vectors using any method well known in the art.

A variety of host-expression vector systems may be utilized to express the silk polypeptide molecules of the invention. Such host-expression systems represent vehicles by which the coding sequences of interest may be produced and subsequently purified, but also represent cells which may, when transformed or transfected with the appropriate nucleotide coding sequences, express silk polypeptide molecule of the invention in situ. These include, but are not limited to microorganisms such as bacteria (e.g., E. coli, B. subtilis, Salmonella) transformed with recombinant bacteriophage DNA, plasmid DNA or cosmid DNA expression vectors containing silk polypeptide coding sequences; yeast (e.g., Saccharomyces and Pichia) transformed with recombinant yeast expression vectors containing silk polypeptide coding sequences; insect cell systems infected with recombinant virus expression vectors (e.g., baculovirus); plant cell systems infected with recombinant virus expression vectors (e.g., cauliflower mosaic virus, CaMV; and tobacco mosaic virus, TMV) or transformed with recombinant plasmid expression vectors (e.g., Ti plasmid) containing silk polypeptide coding sequences; and mammalian cell systems (e.g., COS, CHO, BHK, 293, 3T3 and NSO cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the silk polypeptide being expressed. For example, when a large quantity of such a protein is to be produced vectors which direct the expression of high levels of products that are readily purified may be desirable. Such vectors include, but are not limited to, the E. coli expression vector pUR278 (Ruther et al., EMBO, 12:1791, 1983), in which the silk polypeptide coding sequence may be ligated individually into the vector in frame with the lacZ coding region so that a fusion protein is produced; and pIN vectors (Inouye & Inouye, Nucleic Acids Res., 13:3101-3109, 1985 and Van Heeke & Schuster, J. Biol. Chem., 24:5503-5509, 1989). The non-silk polypeptide portion of the fusion products expressed can then readily be removed.

In an insect system, Autographa californica nuclear polyhedrosis virus (AcNPV) is used as a vector to express foreign genes. The virus grows in Spodoptera frugiperda cells. The silk polypeptide coding sequence may be cloned individually into non-essential regions (for example the polyhedrin gene) of the virus and placed under control of an AcNPV promoter (for example the polyhedrin promoter).

The present invention is also applicable to silk polypeptides derived from conditioned media recovered from mammalian cell cultures that have been engineered to produce the desired silk polypeptides as secreted proteins. Mammalian cell lines capable of producing the subject proteins can be obtained by cDNA cloning, or by the cloning of genomic DNA, or a fragment thereof, from a desired cell as described by Sambrook et al., Molecular Cloning: A Laboratory Manual, 2d Edition, Cold Spring Harbor Laboratory Press (1989). Examples of mammalian cell lines include, but are not limited to, BHK (baby hamster kidney cells), CHO (Chinese hamster ovary cells) and MAC-T (mammary epithelial cells from cows).

In mammalian host cells, a number of viral-based expression systems may be utilized to express an silk polypeptide of the invention. In cases where an adenovirus is used as an expression vector, the silk polypeptide coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the silk polypeptide in infected hosts (e.g., see Logan & Shenk, Proc. Natl. Acad. Sci. USA, 81:355-359, 1984). Specific initiation signals may also be required for efficient translation of inserted silk polypeptide coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bitter et al., Methods in Enzymol., 153:516-544, 1987).

In addition, a host cell strain may be chosen which modulates the expression of the silk polypeptide sequences, or modifies or processes, e.g., glysosylates or cleaves, the silk polypeptide in the specific fashion desired. Different host cells have characteristic and specific mechanisms for the post-translational processing and modification of proteins and gene products. Appropriate cell lines or host systems can be chosen to ensure the correct modification and processing of the silk polypeptide expressed.

For long-term, high-yield production of silk polypeptides, stable expression is preferred. For example, cell lines which stably express the silk polypeptide may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the silk polypeptide.

A number of selection systems may be used, including but not limited to, the herpes simplex virus thymidine kinase (Wigler et al., Cell, 11:223, 1977), hypoxanthineguanine phosphoribosyltransferase (Szybalska & Szybalski, Proc. Natl. Acad. Sci. USA, 48:202, 1992), and adenine phosphoribosyltransferase (Lowy et al., Cell, 22:8-17, 1980) genes can be employed in tk⁻, hgprt⁻ or aprt⁻ cells, respectively. Also, antimetabolite resistance can be used as the basis of selection for the following genes: dhfr, which confers resistance to methotrexate (Wigler et al., Natl. Acad. Sci. USA, 77:357, 1980 and O'Hare et al., Proc. Natl. Acad. Sci. USA, 78:1527, 1981); gpt, which confers resistance to mycophenolic acid (Mulligan & Berg, Proc. Natl. Acad. Sci. USA, 78:2072, 1981); neo, which confers resistance to the aminoglycoside G-418 (Wu and Wu, Biotherapy, 3:87-95, 1991; Tolstoshev, Ann. Rev. Pharmacol. Toxicol., 32:573-596, 1993; Mulligan, Science, 260:926-932, 1993; and Morgan and Anderson, Ann. Rev. Biochem., 62: 191-217, 1993; and May, TIB TECH, 11(5):155-2 15, 1993); and hygro, which confers resistance to hygromycin (Santerre et al., Gene, 30:147, 1984). Methods commonly known in the art of recombinant DNA technology may be routinely applied to select the desired recombinant clone, and such methods are described, for example, in Ausubel et al. (eds.), 1993, Current Protocols in Molecular Biology, John Wiley & Sons, NY; Kriegler, 1990, Gene Transfer and Expression, A Laboratory Manual, Stockton Press, NY; in Chapters 12 and 13, Dracopoli et al. (eds), 1994, Current Protocols in Human Genetics, John Wiley & Sons, NY; and Colberre-Garapin et al., J. Mol. Biol., 150:1, 1981, which are incorporated by reference herein in their entireties.

The expression levels of a silk polypeptide can be increased by vector amplification (for a review, see Bebbington and Hentschel, 1987, The use of vectors based on gene amplification for the expression of cloned genes in mammalian cells in DNA cloning, Vol. 3. Academic Press, New York). When a marker in the vector system expressing silk polypeptide is amplifiable, increase in the level of inhibitor present in culture of host cell will increase the number of copies of the marker gene. Since the amplified region is associated with the silk polypeptide gene, production of the silk polypeptide will also increase (Crouse et al., Mol., Cell. Biol., 3:257, 1983).

The host cell may be co-transfected with two or more expression vectors of the invention, for example, two or more expression vectors encoding different silk polypeptides.

Once a silk polypeptide of the invention has been produced by recombinant expression, it may be purified by any method known in the art for purification of a polypeptides, in particular, silk polypeptides, for example, by chromatography (e.g., ion exchange, affinity, or sizing column chromatography), centrifugation, differential solubility, or by any other standard techniques for the purification of proteins.

### 6.4.2 Silk Polypeptides from Transgenic Animals

Silk polypeptides suitable for use in the present invention, may be extracted from mixtures comprising biological fluids produced by transgenic non-human animals, preferably transgenic non-human mammals. Transgenic animals useful in the invention are animals that have been genetically modified to secrete a target silk polypeptide in, for example, their milk or urine. The methods of the invention are applicable to biological fluids from any transgenic animal capable of producing a recombinant silk polypeptide. Preferably, the biological fluid is milk, urine, saliva, seminal fluid, or blood derived from a transgenic mammal. Preferred mammals are rodents, such as rats and mice, ruminants including cattle such as cows and goats, sheep, and pigs. Preferably, the animal is a goat. See U.S. Pat. No. 5,907,080, hereby incorporated by reference in its entirety. The transgenic animals useful in the invention may be produced as described in PCT publication No. WO 99/47661 and U.S. Patent Publication No. 20010042255, both incorporated by reference herein in their entireties. See, also, the teaching provided in the non-limiting examples, presented below.

### 6.4.3 Silk Polypeptides from Transgenic Plants

The present invention can also be applied to silk polypeptides originating from mixtures comprising plant extracts. Several methods are known in the art by which to engineer plant cells to produce and secrete a variety of heterologous polypeptides (see, for example, Esaka et al., Phytochem. 28:2655-2658 (1989); Esaka et al., Physiologia Plantarum 92:90-96 (1994); Esaka et al., Plant Cell Physiol. 36:441-446 (1995) and Li et al., Plant Physiol. 114:1103-1111 (1997)). Transgenic plants have also been generated to produce spider silk. Scheller et al., Nature Biotech. 19:573 (2001); see also PCT Publication WO 01/94393 A2 (hereby incorporated by reference).

Examples of highly suitable nucleic acid molecules encoding regulatory regions that can, for example, be utilized in expressing a silk polypeptide of the invention in plants and plant cells include, but are not limited to endosperm specific promoters, such as that of the high molecular weight glutenin (HMWG) gene of wheat, prolamin, or ITR1, or other suitable promoters available to the skilled person such as gliadin, branching enzyme, ADFG pyrophosphorylase, patatin, starch synthase, rice actin, and actin, for example.

Other suitable promoters include, for example, the stem organ specific promoter gSPO-A, the seed specific promoters Napin, KTI 1, 2, & 3, beta-conglycinin, beta-phaseolin, heliathin, phytohemaglutinin, legumin, zein, lectin, leghemoglobin c3, ABI3, PvAlf, SH-EP, EP-C1, 2S1, EM 1, and ROM2.

Constitutive promoters, such as CaMV promoters, including CaMV 35S and CaMV 19S can also be used. Other examples of constitutive promoters include Actin 1, Ubiquitin 1, and HMG2.

In addition, a suitable regulatory region for use in expressing a silk polypeptide of the invention may be one which is environmental factor-regulated such as promoters that respond to heat, cold, mechanical stress, light, ultra-violet light, drought, salt and pathogen attack. The regulatory region utilized can also be one which is a hormone-regulated promoter that induces gene expression in response to phytohormones at different stages of plant growth. Useful inducible promoters include, but are not limited to, the promoters of ribulose bisphosphate carboxylase (RUBISCO) genes, chlorophyll a/b binding protein (CAB) genes, heat shock genes, the defense responsive gene (e.g. phenylalanine ammonia lyase genes), wound induced genes (e.g., hydroxyproline rich cell wall protein genes), chemically-inducible genes (e.g., nitrate reductase genes, gluconase genes, chitinase genes, PR-1 genes etc.), dark-inducible genes (e.g., asparagine synthetase gene as described by U.S. Pat. No. 5,256,558), and developmental-stage specific genes (e.g., Shoot Meristemless gene, ABI3 promoter and the 2S1 and Em 1 promoters for seed development (Devic et al., 1996, Plant Journal 9(2):205-215), and the kin1 and cor6.6 promoters for seed development (Wang et al., 1995, Plant Molecular Biology, 28(4):619-634). Examples of other inducible promoters and developmental-stage specific promoters can be found in Datla et al., in particular in Table 1 of that publication (Datla et al., 1997, Biotechnology annual review 3:269-296).

Exudates produced by whole plants or plant parts may be used in the methods of the present invention. The plant portions for use in the invention are intact and living plant structures. These plant materials may be distinct plant structures, such as shoots, roots or leaves. Alternatively, the plant portions may be part or all of a plant organ or tissue, provided the material contains the biofilament protein to be recovered.

Having been externalized by the plant or the plant portion, exudates are readily obtained by any conventional method, including intermittent or continuous bathing of the plant or plant portion (whether isolated or part of an intact plant) with fluids. Preferably, exudates are obtained by contacting the plant or portion with an aqueous solution such as a growth medium or water. The fluid-exudate admixture may then be subjected to the purification methods of the present invention to obtain the desired silk polypeptide. The proteins may be recovered directly from a collected exudate, preferably guttation fluid, or from a whole plant, or a portion thereof.

Extracts useful in the invention may be derived from any transgenic plant capable of producing a recombinant silk polypeptide. Preferred for use in the methods of the present invention are plant species representing different plant families, including, but not limited to, monocots such as ryegrass, alfalfa, turfgrass, eelgrass, duckweed and wilgeon grass; dicots such as tobacco, tomato, rapeseed, azolla, floating rice, water hyacinth, and any of the flowering plants. Other preferred plants are aquatic plants capable of vegetative multiplication, such as Lemna and other duckweeds that grow submerged in water, such as eelgrass and wilgeon grass. Water-based cultivation methods such as hydroponics or aeroponics are useful for growing the transgenic plants of interest, especially when the silk protein is secreted from the plant's roots into the hydroponic medium from which the protein is recovered.

The plant used in the present invention may be a mature plant, an immature plant such as a seedling, or a plant germinating from a seed. According to the methods of the invention, the recombinant polypeptide is recovered from an exudate of the plant, which may be a root exudate, guttation fluid oozing from the plant via leaf hydathodes, or other sources of exudate, regardless of xylem pressure. The proteins may be exited or oozed out of a plant as a result of xylem pressure, diffusion, or facilitated transport (i.e., secretion).

### 6.5. Recovery of Silk Polypeptides from Expression Systems and Biofilament Formation

Methods for the recovery of silk polypeptides from biological fluids are found in PCT Application No. ______ claiming priority to U.S. Provisional Application No. 60/347,471, filed Jan. 11, 2002, which are each hereby incorporated by reference in their entireties. Methods of forming biofilaments from silk polypeptides are described in PCT Application No. ______ claiming priority to U.S. Provisional No. 60/347,510, filed Jan. 11, 2002, and to U.S. Provisional No. 60/408,530, filed Sep. 4, 2002, which are each hereby incorporated by reference in their entireties.

### 7. ILLUSTRATIVE EXAMPLES

The following examples are meant to illustrate the principles and advantages of the present invention. They are not intended to be limiting in any way.

### 7.1. Example 1

### Silk Polypeptides Expressed in Cell Culture

### 7.1.1. Generation of Expression Vectors Encoding Silk Polypeptides with Sequences Derived from Two Spider Species—N. clavipes and A. diadematus

Truncated synthesis has been a limiting factor in expressing silks of high molecular weight size in E. coli and Pichia. Thus, we wanted to evaluate if mammalian cell systems were capable of efficiently overcoming this limitation. As a first step towards this goal, the native sequences encoding the dragline silks have been cloned. Partial cDNA clones encoding the two protein components of the dragline silk have been isolated and characterized from two species of orb-web weaving spiders (A. diadematus and N. clavipes; Xu & Lewis, Proc. Natl. Acad. Sci. 87:7120-7124 (1990); Hinman & Lewis, J. Biol. Chem. 267:19320-19324 (1992)). The sizes of the mRNAs have been determined to be approximately 12 kb and 11.5 kb respectively (Xu & Lewis, Proc. Natl. Acad. Sci. 87:7120-7124 (1990); Hinman & Lewis, J. Biol. Chem. 267:19320-19324 (1992)). Dragline silk genes encode proteins that contain iterated peptide motifs (Hinman et al., Trends in Biotech. 18:374-379 (2000)). They exhibit a pattern of alternating Ala-rich, crystal-forming blocks (ASAAAAAA blocks) and Gly-rich amorphous blocks (GGYGPG, (GPGQQ)ₙ) of similar size. On the basis of physical studies, the crystal-forming blocks have been assigned to specific highly ordered β-sheet structures that impart the silk fiber's mechanical properties (Hayishi et al., Int. J. Biol. Macro. 24:271-275 (1999); Gosline et al., J. Exp. Biol. 202:3295-3303 (1999)). The amorphous domains have been implicated in the formation of a β-turn spiral conformation and provide elasticity (Hayishi & Lewis, Science 287:1477-1479 (2000)). The C-terminal domains of the dragline silks are non-repetitive and show high homology amongst various spider species studied so far. They also contain a highly conserved Cys residue that may be involved in inter-polypeptide disulfide cross-linking (Guerette et al., Science 272: 112-115 (1996)).

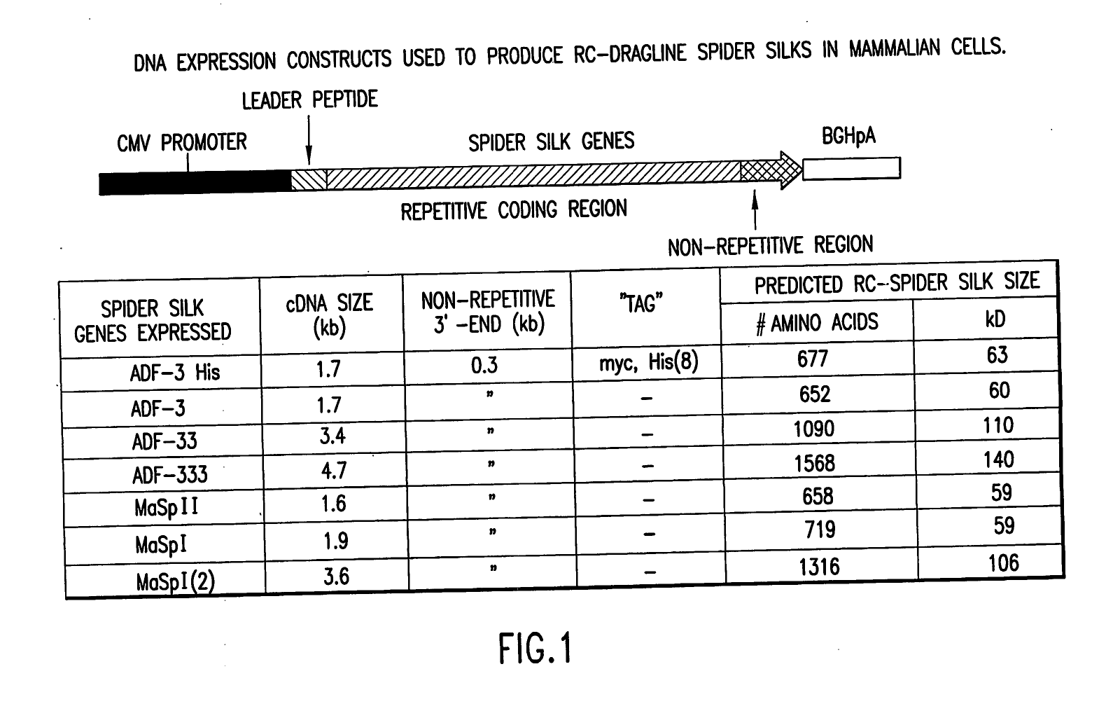

We generated two series of constructs for expression of recombinant (rc)-spider silk proteins in mammalian epithelial cells using spider dragline silk cDNAs: one series containing the MaSpI or MaSpII cDNAs (Xu & Lewis, Proc. Natl. Acad. Sci. 87:7120-7124 (1990)) and a second series containing the ADF-3 cDNA (Guerette et al., Science 272: 112-115 (1996)). In addition, expression vectors were generated containing multimers of the dragline cDNAs (ADF-33 (two repetitive units), ADF-333 (three repetitive units), and MaSpI (2) (two repetitive units)), in which the multimerized units consist of the repetitive coding regions of the spider silks, in order to produce polynucleotides that encode polypeptides of similar size to those found in the spider major ampullate silk gland. Constructs containing up to ten repetitive units can also be generated. In these constructs the carboxyl-terminus was similar to the other cassettes, i.e., contained the 0.3 kb non-repetitive domain (FIG. 1). An additional construct for ADF-3 was prepared that contained a c-myc epitope, in frame after the 0.3 kb C-terminus, and a six-histidine tag to facilitate detection and purification, respectively (FIG. 1). In all cases, the spider silk sequences were under the transcriptional control of a strong constitutive promoter followed by the murine Ig-kappa secretion leader sequence allowing for efficient protein trafficking and secretion of the expressed recombinant spider silks from the epithelial cells.

### 7.1.1.1. Plasmid Construction

All molecular manipulations were carried out following standard procedures (Sambrook et al., Molecular Cloning: A Laboratory Manual, 2d Edition, Cold Spring Harbor Laboratory Press (1989)). All DNA cloning manipulation were performed using E. coli STBII competent cells (Canadian Life Science, Burlington, ON, Canada). Restriction and modifying enzymes were purchased from New England Biolabs (Mississauga, ON, Canada) unless otherwise specified. Construct integrity was verified using DNA sequencing analysis provided by Queens University (Kingston, ON, Canada) or McMaster University (Hamilton, ON, Canada). Primers were synthesized by Dalton Chemical In (North York, ON, Canada). PCR was performed using Ready-To-Go PCR beads (Pharmacia Biotech, Baie d'Urfe, PQ, Canada) or Dynazyme kit (MJ Research, MA). In all expression vectors constructed, the spider silk sequences were under the transcriptional control of a strong constitutive promoter followed by a secretion leader in order to direct efficient trafficking and secretion of recombinant proteins from the epithelial cells. ADF-3 H is contains an in-frame carboxyl-terminal fusion with a c-myc epitope and a six-Histidine tag to facilitate detection and purification, respectively.

### 7.1.1.2. Construction of ADF-3 Vectors

The ADF-3 polynucleotide sequence was PCR amplified from the plasmid BLSK-ADF-3 (Guerette et al., Science 272:112-115 (1996); provided by Dr. Goseline). Two primers (primer 1: 5′-CGTACGAAGCTTATGCACGAGCCGGATCTG-3′ (SEQ ID NO:30); primer 2:5′-ATTAACTCGAGCAGCAAGGGCTTGAGCTACAGA-3′ (SEQ ID NO:31) were designed according to ADF-3 sequences (Guerette et al., Science 272:112-115 (1996)). Primer 1 contains a Hind m site and primer 2 was designed to incorporate an Xho I site. The PCR product was digested with Hind III and Xho I restriction enzymes and DNA fragments were purified using QiexI matrix (Qiagen, Chatsworth, Calif., USA) and cloned into the pSecTag-C vector (Invitrogen, Calif., USA) between the Hind III and Xho I sites. The integrity of the final expression cassette was confirmed by sequencing analysis.

The ADF-3+ H is construct was modified in order to remove the myc tag, His sequences, and a 15 amino acid non-silk sequence present at the N-terminal. A linker containing an Xho I overhang (linker 1: 5′-TCGAGCTTGATGTTT-3′ (SEQ ID NO:32)) was cloned into the ADF-3 His expression cassette between the Xho I and Pme I sites. The 15 amino acid non-silk sequence at the 5′ end of the vector were removed by inserting a linker (linker 2: 5′-CAGGATCTGGACAACAAGGACCCGGACAACAAGGACCCGGACAACAAGGAC CCGGACAACAAGGACCATATGGACCCGGTGCATCCGCCGCAGCAGCAGCCGC TGGAGGTTATGGACCCGGATCTGGACAACAAGGACCCAGCCAACAAGGACCTGG-3′ (SEQ ID NO:33)) into the above vector between the Sfi I and Msc I sites.

To construct the ADF-33 and ADF-333 vectors, the ADF-3 coding region was first released (Msc I and Pvu II: 1.4 kb) and subcloned into the same vector between the Msc I and Pvu II site. Using this procedure, two or three copies of the ADF-3 coding region were inserted into the vector. The new vectors formed by this procedure contained two (ADF-33) or three (ADF-333) copies, respectively, of the ADF-3 sequence.

### 7.1.1.3. Construction of MaSpI Vector

The MaSpI sequence was isolated from the bluescript-MaSp1 plasmid (Xu & Lewis, Proc. Natl. Acad. Sci. 87:7120-7124, 1990; provided by Dr. Lewis). MaSpI vector was constructed in three steps. First, the 3′-end was modified with the addition of a Pme I site after the stop codon (position: 3065 bp) by inserting a linker (5′-CTAGGTTAAGTTTAAACG-3′ (SEQ ID NO:34)) in between the Avr II and Bam HI sites. A 2 kb Hind III/Pme I MaSpI insert was released and cloned into the Hind III/Pme I sites of pSecTag. In order to clone the MaSpI cDNA in frame with the Ig-kappa signal peptide the following modifications were performed. First, the MaSpI vector was digested with Stu I and self-ligated, leaving only 374 bp of the 5′-end of the MaSpI gene (pMaSpI/Stu I). This vector was then used to amplify a fragment containing the 5′-end of MaSpI in frame with the signal peptide. The fragment was amplified by PCR (primer 1: 5′-CAGGTTCCACTGGTGACGCGGCCCAAGGGGCCCAAGGGGCAGGTGCAGCAGCAGCAGCA-3′ (SEQ ID NO:35); primer 2: 5′-GAACCCAGAGCAGCAGTACCCATAG-3′ (SEQ ID NO:36), filled in with T4 DNA polymerase and phosphorylated with polynucleotide kinase. The resulting PCR product contains a Hind III site, in frame with the signal peptide at the 5′ end and Stu I site at the 3′ end. The PCR product was subcloned into the original MaSpI construct between Hind III and the Stu I site located next to the Hind III site using a Stu I partial digestion.

To construct a vector with more than one coding region of MaSpI, the MaSpI vector was digested with Bbs I and the ends were filled in using T4 DNA polymerase in the presence of dNTPs. The MaSpI coding sequence was released with Sac I and cloned into the MaSpI vector between the Sac I and a blunt ended Apa I site. The Apa I site was blunt-ended using T4 DNA polymerase prior to cloning.

### 7.1.1.4. Construction of MaSpII Vector

The MaSpII cDNA sequence was isolated from the plasmid bluescript-MaSp2 (Xu & Lewis, Proc. Natl. Acad. Sci. 87:7120-7124 (1990)). This plasmid was modified at the 5′ end, in order to introduce an Apa I site, by digesting with Bam HI followed by Mug Bean Exonuclease treatment. A linker (primer 1: 5′-AGCGGGCCCGCTCTTC-3′ (SEQ ID NO:37); primer 2: 5′-GAAGAGCGGGCCC-3′ (SEQ ID NO:38)) was cloned into the Sap I site, generating an Apa I site. A second linker (primer 1: 5′-GCAGCAGCAG-3′ (SEQ ID NO:39); primer 2: 5′-GGGCTGCTGCTGCGGCC-3′ (SEQ ID NO:40)) was then cloned in between the Apa I and Sap I sites, allowing the 5′ end of MaSpII to be in frame with the ORF of the pSecTag secretion signal sequence. The 3′ end was modified to introduce a Pme I site by inserting a linker (primer 1: 5′-TGAAATTTCG-3′ (SEQ ID NO:41); primer 2: 5′-AATTCGAAATTTCATGCA-3′ (SEQ ID NO:42)) in between the Eco RI and Nsi I sites. The vector was then digested with Nae I and Eco RV, to remove an Apa I site, and re-circularized. The final construct was digested with Apa I and Pme I and the 2 kb MaSpII insert was cloned into the MaSp1 vector between Apa I and Pme I.

### 7.1.2. Expression of Silk Polypeptides

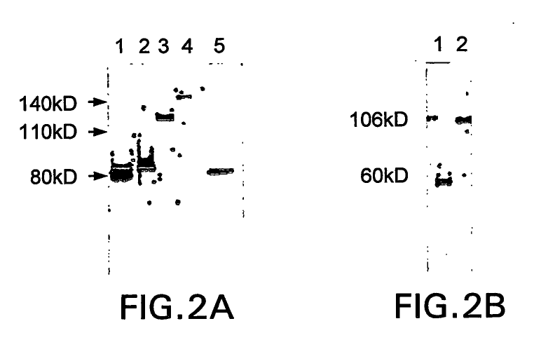

Two mammalian cell lines (MAC-T and BHK cells), known for their ability to secrete complex proteins, were chosen as expression systems. MAC-T cells (Huynh et al., Exp. Cell Res. 197:191-199 (1991)) are mammary epithelial cells that were selected primarily for two reasons: (a) they are epithelial cells, similar to the cell type that expresses the silk proteins in the spider glands (Lucas, Discovery 25: 20-26 (1964)), and (b) they mimic bovine lactation, thereby providing preliminary information in terms of the capacity of mammary epithelial cells to efficiently secrete soluble spider silks. This information is useful when establishing methodologies for the production of recombinant silk polypeptides in the milk of transgenic animals. Analysis of media from stable transfectants of ADF-3, MaSpI, and MaSpII constructs using Western blotting analysis resulted in prominent immuno-reacting bands of the expected molecular weight (FIG. 2A: lanes 1, 2, and 5; FIG. 2B: lane 1).

The first step towards exploring the relationship between spider silk protein size and mechanical properties was to evaluate the ability of the mammalian epithelial cells to produce recombinant spider silk polypeptides of high molecular weight resembling the size of silk proteins observed in the spider's silk gland (Fahnestock et al, Reviews Mol. Biotech. 74:105-119 (2000)). Analysis of conditioned media showed the presence of rc-spider silk proteins of the predicted sizes (˜110 kDa and ˜140 kDa protein; FIG. 2A: lanes 3 and 4; FIG. 2B: lane 2) produced from concatemers of ADF-3 (ADF-33 and ADF-333) and a dimer of MaSpI, respectively. In all cases, the different expression vectors used enabled the secretion of soluble silk proteins in the media. Distinct spider silk proteins of sizes ranging from 120 kDa, 150 kDa, 190 kDa, 250 kDa, up to 750 kDa have been found in the ampullate gland of Nephilia clavipes (Fahnestock et al, Reviews Mol. Biotech. 74:105-119 (2000)).

The expression levels of the secreted 110 and 140 kDa spider silk proteins from BHK cells were much lower than the 60 kDa monomer. This may be attributed to inefficient transcription due to high secondary structure, insufficient secretion of the larger proteins, low number of copies of the construct being transfected, or limitations in the cell translational machinery. It has been shown that during silk synthesis, the spider produces gland specific pools of tRNAs for glycine and alanine in order to meet the increased demand for limiting amino acids (Candelas et al., Dev. Biol. 140:215 (1990)). It is possible that due to the unique amino acid composition of the silk proteins (for example: MaSpII: 32% glycine, 16% alanine) the aminoacyl-tRNA pools of the epithelial cells grown in vitro are depleted. When screening clones for the expression of the multimerized genes, we observed the expression of proteins with distinct molecular weights, both larger and smaller than the predicted molecular weights (data not shown). The pattern of expression was different than the “ladder” effect observed with the monomers during scale up production (see below). We hypothesized that this may be due to rearrangements/recombination of the construct, after long-term culture, due to the large size and highly repetitive nature of the cDNAs, similarly reported previously (Prince et al., Biochemistry 34:10879-10885 (1995)).

### 7.1.2.1. Transfection and Selection of Stable Cell Lines

MAC-T (Huynh et al., Exp. Cell Res. 197:191-199 (1991)) or BHK cells were seeded at a density of 5×10⁵ cells per 100 mm dish. On the following day, cells were transfected with the spider silk gene plasmids or with the empty vector (without the spider silk cDNA). Ten μg of the plasmid DNA was diluted into 0.25 ml of DMEM and mixed with an equal volume of Lipofectamine (Canadian Life Science; 20 μg of lipid in 0.25 ml DMEM). The mix was vortexed for 10 sec, and the complexes were allowed to form for 30 min at room temperature. The volume was increased to 4 ml with DMEM and the lipid-DNA mixture was applied to the cells and allowed to incubate for 16-20 h at 37° C./5% CO₂. The cells were then cultured for another 24 h in fresh medium containing 10% FCS. Subsequently, the cells were selected in the same media containing 100 μg/ml hygromycin B. Colonies surviving selection were picked after 7-8 days following transfection and expanded further. In general, the results indicated that under the culture conditions tested, BHK cells transfected with the spider silk constructs expressed higher amounts of the rc-ADF-3 proteins than the MAC-T cells.

### 7.1.2.2. Hollow Fiber System for Cell Culture

Unisyn's CELL-PHARM® System 2500™ hollow fiber cell culture system was used for the production and continual recovery of mammalian secreted rc-spider silk proteins. Typical production of rc-spider silk protein using the hollow fiber system was achieved for up to 3 months.

### 7.1.2.3. Generation of Polyclonal Antibodies against Silk Polypeptides

Antibodies were raised in rabbits against both purified rc-spider silk protein (BHK derived material) and synthetic peptides designed based on sequences of N. clavipes and A. diadematus. Peptide synthesis, conjugation, immunization, bleeding, and serum preparations were carried out by Strategic BioSolutions (Ramona, Calif.). The immunizing peptide sequences were anti-MaSpII, GLGSQGAGRGGQGAGA-NH₂, anti-ADF-3, ARAGSGQQGPGQQGPG-NH₂.

### 7.1.2.4. Detection of rc-Spider Silk Polypeptides in Media and Purified Fractions

Quantitation of rc-spider silk polypeptides in conditioned media involved SDS-PAGE and immunologic evaluation (Western blotting analysis). Serum free conditioned media was harvested from cells at 70-80% confluency at 24 hrs. An aliquot of 20 μl was loaded onto 8-16% Tris-Glycine gels (Novex, Invitrogen), electrophoresed and transferred by electroblotting onto nitrocellulose membrane. Rc-spider silk immunoreacting proteins on the membrane were detected using rabbit polyclonal antibodies raised against ADF-3 or MaSpI (1:5000 dilution) and goat anti-rabbit horseradish peroxidase conjugated 2nd antibody. Detection was performed according to the manufacturer's protocol using enhanced chemiluminescence (ECL) detection (Amersham/Pharmacia). For silver stain analysis, gels were stained using GelCode SilverSNAP (pierce, IL) kit, as described by the manufacturer. Samples were prepared by adding 10 M urea to a final concentration of 6 M, loading buffer containing β-mercaptoethanol and heating for 5 min at 95° C. prior to loading. In the absence of urea, aberrant migration of rc-spider silk protein was observed.

### 7.1.3. Large-Scale Production of Silk Polypeptides in Cell Culture

Production of 25-50 mg/L (˜20 μg/10⁶ cells/day) of ADF-3 His and ADF-3, rc-spider silk protein was achieved in BHK cells with over 12 g of material purified from conditioned cultured media. A correlation was observed between the age of the reactor (˜3 months) and the appearance of lower molecular weight spider silk proteins. The appearance of this protein “ladder” was probably due to termination errors of protein synthesis. Translational pausing, resulting in heterogeneous protein expression, has been reported in N. clavipes (Gosline et al., J. Exp. Biol. 202:3295-3303 (1999); Arcidiacono et al., Appl. Microbiol. Biotechnol. 49:31-38 (1998) and B. mori (Lizardi et al., Proc. Natl. Acad. Sci. USA 76:6211-6215 (1979)). Similar protein “ladder” effects were observed in cell lines expressing ADF-3 His when antibodies to ADF-3 were used. However, the protein “ladder” was not detectable when antibodies against the myc epitope where used for detection, since it would recognize only intact-full length spider silk proteins. In addition, when silk protein was purified using the His affinity tail only a single protein band was detected, indicating that the ladder was due to deletions at the carboxyl end.

### 7.1.4. Purification of Silk Polypeptides from Cell Culture

ADF-3 was recovered from conditioned culture media by precipitation with 15-20% ammonium sulfate for an enrichment of at least 50% in a single step. The precipitated proteins, including ADF-3, were readily dissolved in aqueous buffer (phosphate buffered saline). Recombinant spider silks produced in E. coli or yeast and precipitated similarly would only be redissolved in strong denaturing solvents such as hexafluoroisopropanol or guanidine hydrochloride (Fahnestock et al, Reviews Mol. Biotech. 74:105-119 (2000)). While not intending to be bound by any particular theory or mechanism of action, the difference in solubility is believed to result from the presence of the carboxyl-terminus in ADF-3 and MaSpII rc-spider silk proteins produced in epithelial cells, suggesting that the more hydrophilic carboxyl-terminus of 100 amino acids (absent in other studies) may increase the solubility of secreted silks.

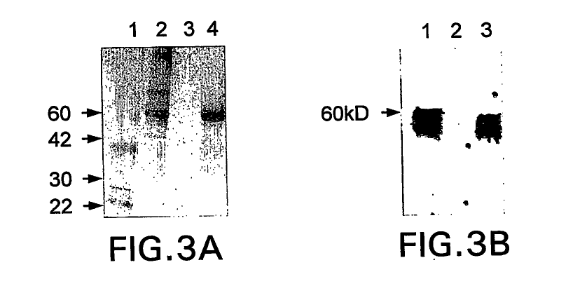

Purified ADF-3 migrated as a major band with an apparent molecular mass of 60 kDa silver stained SDS-P AGE gels under reducing conditions (FIG. 3A: lane 4) and was recognized by ADF-3 specific antibodies (FIG. 3B: lane 3). Purities of rc-spider silk achieved ranged from 80-90%.

The identity of the purified ADF-3 protein was confirmed by N-terminal sequencing. It exhibited identity to the first 6 residues, confirming the predicted amino acid sequence and cleavage of the leader peptide at the expected site. Amino acid analysis of the purified ADF-3 protein further confirmed the identity and purity of the protein.

### 7.1.4.1 Methods of Purification of Silk Polypeptides from Cell Culture

The following protocols describe methods of purification of the silk polypeptides from cell culture media.

### 7.1.4.1.1. ADF-3-His Purification

The conditioned cell culture media was adjusted to contain 6 M urea and then loaded onto a Ni-NTA column (Qiagen, Chatsworth, Calif., USA) and processed as described by the manufacturer. Bound proteins were eluted using wash buffer containing 100 mM imidazole. Eluted fractions were analyzed as described above.

### 7.1.4.1.2. Purification of Unlabeled ADF-3

Conditioned culture media was filtered using a 0.45 μm filter, brought to a final concentration of 20% (w/v) ammonium sulfate and incubated for 1 hour at 4° C. Precipitated proteins were recovered by centrifugation at 20,000 g at 4° C. for 1 hour. The protein pellet was gently resuspended in buffer A (20 mM glycine, pH 10) and insoluble material was removed by a brief centrifugation. The pH of the sample was adjusted to 10 using NaOH (10 N), and conductivity was adjusted to 1.2 mS by diluting the sample with buffer A. An anion exchange column of 5×11 cm was packed with POROS HQ50 resin (PE Biosystems, USA) and equilibrated with 10 column volumes of buffer A. The sample was loaded onto the column at a flow rate of 100 mL/h. The column was then washed with S column volumes of buffer A and ADF-3 protein eluted using 3 column volumes of buffer A containing 0.15 M NaCl.

### 7.1.4.1.3. Purity Assessment of Silk Polypeptides

The purity of the rc-silk protein was analyzed using silver staining, RP-HPLC, and amino acid composition. The peak containing ADF-3 protein on RP-HPLC was identified by Western blot analysis. Purity was estimated using peak area integration. Amino acid composition was performed as previously described (Heinrikson et al., Anal. Biochem. 136:65 (1984)).

### 7.1.4.1.4. Quantitation of Purified rc-Spider Silk Proteins

Purified material was quantitated using the extinction coefficient method (at 280 μm) (Gill et al., Anal. Biochem. 182:319 (1989)).

### 7.1.4.1.5 Spin Dope Preparation and Biofilament Testing

The purified material from above can be concentrated to spin dopes containing 5%, 10 up to 40 into suitable buffers and reducing the volume for example by ultrafiltration using 10,000 MWCO membranes (Millipore, Bedford, Mass.).

For fiber testing, denier determination was done using a Vibramat M (TEXTECHNO Herbert Stein GMBH Co., Monchegladbach, Germany) or by polarizing light microscopy. Mechanical testing was performed using the Instron Model 55R4201 (Instron Corp., Canton, Mass.) at 23° C. and 50% relative humidity.

Additional detailed methodology can be found in Lazaris et al., Science 295:472-476 (2002), incorporated by reference herein in its entirety.

### 7.2. Example 2

Techniques to generate transgenic animals by the introduction of a recombinant DNA into zygotes, fetal cells, or oocytes ate well known (reviewed by Wall, Theriogenology 45:57-68, 1996). Methods to develop transgenic animals carrying a gene fused to a tissue-specific promoter, such as a milk-specific promoter (e.g., β-casein, αS1-casein, αS2-casein, β-casein, κ-casein, β-lactoglobin, and α-lactalbumin), are also known (WO 93/25567). The use of transgenic animals carrying transgenes, such as the ones discussed in the invention, makes it possible to produce desired polypeptides in those animals. These polypeptides can be produced in larger quantities and with less expense than those produced using more traditional methods of protein production in microorganisms or animal cells. Once transgenic animals are generated, their offspring can be used in efficient, tissue-specific production of desired polypeptides.

### 7.2.1. Transgenic Goats: Mammary Gland Specific Expression Vectors

Based on the mouse Whey Acidic Protein (WAP) promoter, zygote production can be generated by pronuclear microinjection of zygotes or by nuclear transfer (see Baldassarre et al., WO 09/698,867 and U.S. Ser. No. 09/040,518). Using this methodology, a male founder animal, for example, a goat, is generated that is transgenic for a nucleic acid construct containing a silk polynucleotide sequence, for example, the ADF-33 or ADF-333 construct, encoding a polypeptide of two, three, or more repetitive units of dragline silk. The transgenic founder animal is used to produce FI generation offspring, which are hormonally induced into lactation. The milk of the transgenic animal is collected and the silk polypeptide is purified and subsequently used for fiber spinning. Alternatively, a female founder can be generated, induced into lactation at young age by hormonal treatment and the produced milk tested for the presence of the silk polypeptides.

Based on the mouse WAP promoter, a transgenic founder animal, for example a goat, can be generated by either pro-nuclear microinjection or nuclear transfer technique (see e.g., U.S. Ser. No. 09/040,518), such that the transgenic animal carries a nucleic acid construct encoding a silk polypeptide, for example the ADF-33 or the ADF-333 construct, encoding a polypeptide of two, three, or more repetitive units of dragline silk, The transgenic animals is induced hormonally into lactation at an early age followed by expression of the silk polypeptide. The milk is collected and the silk polypeptide is purified and subsequently used for fiber spinning.

Based on the mouse WAP promoter, a transgenic female founder can be generated using the nuclear transfer technique. The transgenic female founder animal is hormonally induced into lactation (average 77 days of age), and high expression (>1.0 g of silk protein per liter of milk) can be confirmed by testing the milk of the transgenic animal for the presence of the expressed silk polypeptide.

Expression vectors can also be made based on β-casein promoter.

Expression vectors can also be made based on urine specific promoters, specifically the uromodulin promoter.

### 7.3. Example 3

### Synchronization and Gonadotrophic Stimulation of Goats to be Used as Donors of Oocytes Recovered by LOPU

Oocytes recovered by this method are to be used either for the production of zygotes which are microinjected with the transgene or to be used in nuclear transfer experiments where they are fused with a cell type which has been genetically modified.

Adult Goats: Adult goats may be subjected to LOPU without any hormonal stimulation. However, higher numbers of oocytes are obtained if donor goats are synchronized and stimulated with gonadotrophins. Synchronization of donor goats may be achieved using established protocols known to those skilled in the art. The following is an example of a synchronization protocol which may be used.

Intravaginal sponges containing 60 mg of medroxyprogesterone acetate are inserted into the vagina of donor goats and left in place for 7 to 10 days, with an injection of 125 μg cloprostenol given 48 hours before sponge removal. Typically, for recovery of immature oocytes, the sponge was left in place until the oocyte collection, while for the recovery of oocytes more advanced in maturation, the sponge is removed up to 48 hours before the oocyte collection.

The priming of the ovaries was achieved using gonadotrophic preparations including follicle stimulating hormone (FSH), equine chorionic gonadotrophic (eCG), and human menopausal gonadotrophic (hMG). Any established regime for superovulation known by those skilled in the art may be used. The following hormonal regimes are examples of methods which may be used. A total dose equivalent to 120 mg of NIH-FSH-P1 is given twice daily in decreasing doses (35 mg/dose on the first day, 25 mg/dose on the second day) starting 48 hours before sponge removal. Alternatively, 70 mg of NIH-FSH-P 1 may be given together with 400 IU of eCG 36 to 48 hours before LOPU. The recovered oocytes are then matured in vitro as described in Section 7.5.

An alternative strategy for the recovery of oocytes is to aspirate oocytes which have been matured in vivo. For this purpose it is essential to control the number of hours between the luteinizing hormone (LH) peak and the time at which the oocytes are collected. This may be achieved by drug-induced depletion of the endogenous LH peak. For example, the FSH/LH contents of the hypophysis may be depleted using gonadotrophic releasing hormone (GnRH) agonists such as buserelin or deslorelin. Alternatively, the hypophysis may be made refractory to hypothalamic GNRH using a GnRH antagonist such as cetrorelix. The desired GnRH agonist/antagonist may be administered by means of repeated injections, or more appropriately, by means of drug release devices such as subcutaneous implants or pumps. The GnRH agonist/antagonist is administered to the donor goats for at least 7 days prior to the start of gonadotrophic stimulation, and the treatment is continued until the LOPU procedure occurs. Follicular development is then stimulated by means of administration of gonadotrophins using a similar protocol as described above. Prepubertal Goats To recover oocytes from prepubertal goats, synchronization is not required. However, for recovering high numbers of oocytes, donor goats may need to be stimulated with gonadotrophic. This may be achieved by applying the same regimes used for superovulation of adult goats, as described above.

### 7.4. Example 4

### Laparoscopic Ovum Pick-Up

Oocytes from donor goats are recovered by aspiration of follicle contents (puncture or folliculocentesis) under laparoscopic observation. The laparoscopy equipment used (commercially available from Richard Wolf, Germany) is composed of a 7 mm telescope, light cable, light source, 7 mm trocar for the laparoscope, atraumatic grasping forceps, and two 5 mm “second puncture” trocars. The follicle puncture set is composed of a puncture pipette, tubing, a collection tube, and a vacuum pump. The puncture pipette is made using a PVC pipette (5 mm external diameter, 2 mm internal diameter) and a 20G short bevel hypodermic needle, which is cut to a length of 5 mm and fixed into the tip of the pipette with instant glue. The connection tubing is made of silicon with an internal diameter of 5 mm, and connected the puncture pipette to the collection tube. The collection tube is a 50 ml centrifuge tube with an inlet and an outlet available in the cap. The inlet is connected to the pipette, and the outlet is connected to a vacuum line. Vacuum is provided by a vacuum pump connected to the collection tube by means of PVC 8 mm tubing. The vacuum pressure is regulated with a flow valve and measured as drops of collection media per minute entering the collection tube, and is usually adjusted to 50-70 drops/minute.

The complete puncture set is washed and rinsed ten times with tissue culture quality distilled water before gas sterilization, and one time with collection medium before use. The collection medium is TCM 199 supplemented with 0.05 mg/ml of heparin and 1% (v/v) fetal calf serum (FCS). The collection tube contained approximately 0.5 ml of this medium to receive the oocytes.

The goats are fasted 24 hours prior to laparoscopy. Anaesthesia is induced by intravenous administration of diazepam (0.35 mg/kg body weight) and ketamine (5 mg/kg body weight), and maintained with isofluorane via endotrachial intubation. The animals are restrained in a cradle position for laparoscopic artificial insemination as described by Evans and Maxwell, Salomon's Artificial Insemination of Sheep and Goats, Sydney: Butterworths (1987). The 3 trocars described above are inserted and the abdominal cavity is filled with filtered air. The ovary surface is visualized and the follicles are punctured by pulling the fimbria in different directions with the grasping forceps. The needle is inserted into the follicle and rotated gently to ensure that as much of the follicle contents as possible are aspirated. After aspiration of 3 to 5 follicles, the pipette and tubing are rinsed using sterile collection media.

### 7.5. Example 5

### Culture and Enucleation of Oocytes Recovered from Goats by LOPU

Oocyte preparation: Cumulus-oocyte complexes (COCs) are recovered from primed follicles by LOPU. The COCs are washed once in 2 ml of M199 containing 0.5% BSA, placed into 501 drops of maturation medium, covered with an overlay of mineral oil (Sigma), and incubated at 38.5° C. to 39° C. in 5% CO₂. The maturation medium consists of M199 supplemented with bLH (0.02 U; Sioux Biochemicals), bFSH (0.02 U; Sioux Biochemicals), estradiol-17 (1 μg/ml; Sigma), sodium pyruvate (0.2 mM; Sigma), kanamycin (50 μg/ml), and 10% heat-inactivated fetal calf serum (ImmunoCorp), goat serum, or estrous goat serum. After 23-24 hours of maturation, the cumulus cells are removed from the matured oocytes by placing the COCs in a 1.5 ml microcentrifuge tube containing 250 μl of EmCare supplemented with hyaluronidase (1 mg/ml), and vortexing for 1-2 minutes. The cumulus cells may be used in subsequent manipulations, for example, gene transfer, as donor cells for oocytes derived from the same animal or a different animal.

The denuded oocytes are washed in EmCare containing 1% FCS and returned to maturation medium. Fifteen to twenty denuded oocytes are placed into a microdrop (50 μl) containing 5 μg of the fluorescent DNA dye Hoeschst 33342 (stock solution 1 mg/ml saline) in 1 ml of EmCare containing 1% FCS. The oocytes are incubated in the Hoeschst-EmCare solution for 20-30 minutes at 30-36° C.

Manipulation of Oocytes: One manipulation drop (150 μl) of Em Care supplemented with 1% FCS is placed into a 100 mm Optics dish (Falcon), centered, and covered completely with mineral oil. Oocytes stained with the Hoeschst dye are placed into the center of the manipulation drop. Each oocyte is picked up using the holding pipette and rotated until the polar body (PB) is visualized between 3- and 6 o'clock. The edge of the oocyte-containing polar body is moved into a fluorescent UV light path and the location of the chromosomes are noted. The oocyte is pulled slightly out of the UV light path, and the cytoplasm in the area containing the chromosomes and polar body is removed using the manipulation pipette. The removed cytoplasm is checked for the presence of chromosomes and the polar body by moving the pipette into the UV light path; the process is repeated until all oocytes are enucleated. The enucleated oocytes are then placed into a droplet of EmCare containing 1% FCS, and overlaid with 2 ml of mineral oil in a Falcon 1008 dish. These dishes are kept on a warm surface (30-36° C.). Alternatively, the enucleated oocytes are returned to the maturation drop if the nuclear transfer procedure is not immediate.

Isolation of Activated Oocytes: Alternatively, if desired, an activated oocyte may be used to carry out the present invention. To activate an oocyte, one would carry out the oocyte preparation and manipulation procedures as described above. Upon observation of the denuded oocytes stained with Hoeschst 33342, oocytes which are in the telophase stage of nuclear maturation are considered to be activated. These oocytes may be selected and fused with a cell to form a fused couplet which does not require further activation.

### 7.6. Example 6

### Transgenes Used for the Generation of Transgenic Goats and the Production of Heterologous or Homologous Silk Polypeptides in Milk, Urine, Seminal Fluid, Saliva, or Blood of the Transgenic Animal

A genetic construct suitable for use in the present invention generally includes the following elements:

(a) a promoter or transcription initiation regulatory unit;

(b) a transcription termination codon;

(c) DNA encoding a useful protein

(d) a naturally-occurring or synthetic sequence encoding a signal polypeptide directing the secretion of the recombinant protein from the cell and

(e) optionally, an insulator element (e.g., chicken β-globin or chicken lysozyme MARS elements) which may result in a gene dosage effect (i.e., more copies of the transgene yield increased protein expression) or may allow for position-independent expression which is a result of the insulating effect from surrounding chromatin.

Conventional molecular biology methods are used to generate and assemble the above elements.

Milk-specific expression of a heterologous or homologous protein: Useful promoters include as I-casein (as described, for example, in U.S. Pat. No. 5,304,489), αS2-casein, β-casein, κ-casein, β-lactoglobulin (as described, for example, in U.S. Pat. No. 5,322,773), α-lactalbumin, and whey acidic protein (WAP). If desired, the promoter may be linked to enhancer elements (such as CMV or SV40) or insulator elements (such as chicken β-globin).

An example of a DNA expression cassette using the WAP promoter, for example, as described in WO 92/22644, and insulator elements operably linked to a heterologous gene (in this case, a gene from a spider encoding components of spider silk) can be used as illustrated in WO 99/47661A2. This genetic construct also includes a transcription termination region. Preferably, the termination region includes a poly-adenylation site at the 3′ end of the gene from which the promoter region of the genetic construct was derived. The heterologous or homologous gene may be either a cDNA or genomic clone containing introns (all or a subset). If the gene is a cDNA clone, the genetic construct preferably also includes an intron which may increase the level of expression of the particular gene. Useful introns, for example, are those found in genes encoding caseins.

Urine-specific expression of a heterologous or homologous protein: Useful promoters for the urine-specific expression of a heterologous or homologous protein are II those disclosed in PCT/US96/08233, and U.S. Pat. No. 5,824,543, such as uroplakins I, II, and III, hereby incorporated by reference. The uroplakin II promoter, for example, has been shown to direct the expression of hGH in the urine of transgenic mice in detectable levels. Other useful promoters include kidney-specific promoters such as rennin and uromodulin.

Constructs harboring the concatemer plus the transcriptional control units can be harbored into plasmid vectors or yeast artificial chromosomes (YACS) or mammalian artificial chromosomes.

### 7.7. Example 7

### Transfer Experiments

In all of the above examples, the genetic construct may be introduced into a cell type of interest, for example; a fetal fibroblast (using, for example, the methods of Cibelli et al., Science 280:1256-1528 (1998)) or cumulus cells (using, for example, the methods of Kato et al., Science 282:2095-2098 (1998)) by a variety of techniques, including electroporation, lipofection, calcium phosphate transfection, viral infection, and microinjection. Preferably the transgene is transfected with a selectable marker so selection of cells containing the transgene may be achieved. Such selection markers include, but are not limited to G418, hygromycin, and puromycin. It may also be desirable for the trans gene to specifically target an area of the genome of the cell by using, for example, the Cre-Iox system (Melton, Bioessays 16:633-638 (1994); Guo et al., Nature 389:40-46 (1997)). In all of the examples described above the selected cell line is used in the subsequent step of fusion with an enucleated LOPU-derived oocyte.

### 7.8. Example 8

### Generation of Transgenic Animals: The Nuclear Transfer Technique

The following example describes generation of transgenic animals utilizing the nuclear transfer technique.

### 7.8.1. Nuclear Transfer (Fusion and Activation) and Culture of the Nuclear Transfer-Derived Embryo Culture

Preparation of donor cells by serum starvation to generate G0 cells: Fetal fibroblasts were isolated from day 27 to day 30 fetuses from the dwarf breed of goat BELE® (Breed Early Lactate Early). The cells are transfected with a construct encoding the silk polypeptide, for example, the ADF-33 or ADF-333 construct, encoding a silk polypeptide of two, three, or more repetitive units. The transfected cells are then used as donor cells in nuclear transfer.

Eight days prior to the nuclear transfer, 2.5×10⁴ donor cells are plated in one well of a 24-well plate in 1.5 ml of complete media (DMEM supplemented with 10% FBS, 0.1 mM mercaptoethanol, and 0.1% gentamycin) and incubated in a humidified atmosphere at 37° C. and 5% CO₂. The next day, fresh complete media is added to the well. Two days later the media is again replaced with fresh media. Four to eight days prior to nuclear transfer, the cells are washed twice, placed into low serum media (DMEM supplemented with 0.5% FBS, 0.1 mM β-mercaptoethanol, and 0.1% gentamycin), and returned to the incubator (37° C. and 5% CO₂ until the day of nuclear transfer. Low serum media is replaced with fresh low serum media every 24-48 hours.

On the day of nuclear transfer the donor cells are prepared as follows. Thirty minutes before they are needed, the cells are rinsed quickly with pre-warmed 0.05% trypsin/EDTA, and incubated with 200 μl of the same solution for 3 minutes in the incubator. The cells are recovered from the well and placed into a cryovial with EmCare supplemented with 1% FCS. The cells are pelleted by centrifugation (875 g for 3 min) and resuspended twice in EmCare supplemented with 1% FCS. The final donor cell suspension (500 μl per ml of EmCare containing 1% FCS) is placed in a 35 mm suspension dish and the cells are used immediately for nuclear transfer.

### 7.8.2. Oocyte Preparation

Cumulus-ooctyes complexes (COCs) are recovered from primed follicles by LOPU as described above.

### 7.8.3. Manipulation of Oocytes

The enucleation of LOPU-derived oocytes is achieved as described above.

### 7.8.4. Fusion

A donor cell is picked up with the manipulation tool and slipped into the perivitelline space. Cell-cytoplast couplets are fused using electrofusion as soon after enucleation of the oocytes as possible. The couplets are moved through dishes containing (i) EmCare supplemented with 1 mg of BSA/ml; (ii) a 1:1 dilution of sorbitol fusion medium (0.25 M sorbitol, 0.1 mM calcium acetate, 0.5 mM magnesium acetate, 0.1% bovine serum albumin) and EmCare; and (iii) sorbitol fusion medium. Groups of four to six couplets are aligned between the electrodes of a BTX fusion chamber (catalog No. 450) in a 100 mm plate containing sorbitol fusion medium. A brief fusion pulse is administered by a BTX and optimizer. A typical pulse of 17 μsec at 2.39 kV/cm (90 V peak) is applied.

The couplets are moved through the sorbitol fusion medium/EmCare solution and the EmCare/BSA solution, and then placed in microdrops of EmCare supplemented with 1% FCS. After all couplets have been exposed to the fusion pulse they are placed into culture drops of the appropriate medium (SOFM according to Tervit et al., J. Reprod. Fertility 30:493-497 (1972); G1 according to Gardner & Lane, Human Reprod., Update 3; 367-382 (1997); or TCM containing 10% fetal calf serum, and incubated at 38.5° C.-39° C. in 5% CO₂, 7% O₂, and 88% N₂.)

After 2-3 hours, the fused couplets are activated using the calcium ionophore and DMAP method of Susko-Parrish et al. (Biol. Reprod. 51:1099-1108 (1994)) or by application of additional electrical pulses (1.26 kV/cm, 80 μsec), followed by incubation in nocodozole or cytochalasin B (Campbell et al., Nature 380:64-66, 1996). After being cultured for 2.5 to 4 hours in DMAP, nocodazole, or cytochalasin B, activated nuclear transfer-derived zygotes are returned to culture drops containing SOFM or G1. Cleavage development (2- to 4-cell stages) is observed at 22 hours (the night before embryo transfer) and 36 hours (the morning of embryo transfer). Nuclear transfer-derived embryos are transferred into synchronized recipients between days 1 and 12 post fusion (day 0=day of fusion).

### 7.8.5. In Vitro Culture

Reconstructed embryos are placed into microdrops of 25 μl of G1 or low phosphate (0.35 mM) SOFM embryo culture medium (Gardner et al., Biol. Reprod. 50:390-400 (1994)) under an oil overlay. After 48-72 hours, cleaved embryos are moved to fresh microdrops of embryo culture medium. On day 4 or 5 (day 0=day of fusion) embryos are moved to microdrops of G2 medium or high phosphate (1.2 mM) SOFM.

### 7.8.6. Embryo Transfer

Nuclear transfer-derived zygotes, or cleaved embryos at the 2- to 8-cell stage are transferred into the oviduct of a synchronized recipient. Morulae and blastocysts are transferred into the uterus of a synchronized recipient. Pregnancies are determined at 30 and 60 days of gestation.

### 7.9. Example 9

### Synchronization of Animals to be Used as Recipients of Nuclear Transfer-Reconstructed Embryos Derived Using Oocytes from LOPU Procedures

Recipients are synchronized by any established regime known by those skilled in the art. They should be observed on standing heat during the day that the oocytes are enucleated. The following hom1onal protocol is one example of a method which may be used. Intravaginal sponges containing 60 mg of medoxyprogesterone acetate are inserted into the vagina of recipient goats and left in place for 7 to 10 days with an injection of 125 μg closprostenol given 48 hours before sponge removal. Sponges are removed and an injection of 400 IU of eCG is administered on the same day as the LOPU takes place.

### 7.10. Example 10

### Transfer of Embryos Reconstructed by Nuclear Transfer Using LOPU-Derived Oocytes to Recipient Goats

Reconstructed nuclear transfer embryos are either incubated for a short period (42-48 hours) or 5 days and then transferred to synchronized recipient goats. The recipient goats are fasted 24 hours prior to surgery. Anesthesia is induced by intravenous administration of diazepam (0.35 mg/kg body weight) and ketamine (5 mg/kg body weight), and maintained with isofluorane via endotrachial intubation.

A laparoscopic exploration is then perfom1ed to confim1 if the recipient had one or more recent ovulations/corpora lutea (CL) present in the ovaries and a normal oviduct and uterus. The laparoscopic exploration is carried out to avoid performing a laparotomy on an animal which has not responded properly to the hom1onal synchronization protocol and to which an embryo should not be transferred. If the short culture period is preferred (overnight following nuclear transfer/fusion), the embryos may be transferred to the oviduct of recipient goats. For this purpose, a mid-ventral laparotomy of approximately 10 cm in length is established, the reproductive tract is exteriorized, and the embryos are implanted into the oviduct ipsilateral to ovulation/s by means of a TomCat catheter threaded into the oviduct from the fimbria.

If embryos are cultured for 5 days, the resulting morula/blastocyst-staged embryos may be transferred to the uterus. For this purpose, a mid-ventral laparotomy of approximately 5 cm in length is established and the uterine horn ipsilateral to the CLs is exteriorized using a surgical clamp under laparoscopic observation. A small perforation is made with an 18G needle in the oviductal third of the horn, and the embryos are then implanted by means of a TomCat catheter threaded into the uterine lumen.

### 7.11. Example 11

### Proteolytic Cleavage Separating the Repetitive Units from the Non-Repetitive Hydrophilic Domain in MaSpII Silk Polypeptide

The following is illustrative of the use of trypsin to cleave near the Arg (R) residue located between the region of repetitive units and the non-repetitive hydrophilic domain in MaSpII silk polypeptide as shown in FIG. 6.

MaSpII silk polypeptide, as expressed in, and purified from goat milk according to the methods described above, was dissolved in 6 M guanidine-HCL and buffer-exchanged in 50 mM glycine, pH 11, using a G25C desalting column. A 300 mg portion of purified MaSpII was adjusted to 1 mg/ml and dialyzed overnight against 100 mM NH₄HCO₃, pH 8 (Ambic buffer). Trypsin, solubilized in Ambic buffer at 1 mg/ml just prior to use, was added in a 6 mL volume to 300 mL of dialyzed MaSpII (0.98 mg/ml) to obtain a protease:protein ratio of 1:50, and the solution was incubated at 37° C. during 4 hour with slow stirring. Ammonium sulfate was slowly added to the cleavage mixture to reach 1.1 M. The solution was gently stirred overnight overnight at 4° C. prior to centrifugation at 30,000 g for 30 min at 4° C. The protein pellet was dissolved in 60 mL 6 M guanidine-HCL and buffer-exchanged in 50 mM glycine, pH 11, using a G25C desalting column. The final quantity of MaSpII-repetitive region was 156 mg, and analysis by RP-HPLC indicated that 95% of the full-length MaSPII polypeptide was cleaved in the cleavage reaction (results not shown).

All publications and patent applications mentioned in this specification are herein incorporated by reference to the same extent as if each independent publication or patent application was specifically and individually indicated to be incorporated by reference.

What is claimed is:

1. An isolated silk polypeptide comprising a plurality of repetitive units and a non-repetitive hydrophilic amino acid domain.

2. The silk polypeptide of claim 1, wherein at least two repetitive units are present in a head-to-tail configuration.

3. The silk polypeptide of claim 1, wherein the repetitive units are present in a head-to-tail configuration.

4. The silk polypeptide of claim 1, wherein at least two repetitive units are present in a head-to-head configuration.

5. The silk polypeptide of claim 1, wherein all the repetitive units are present in a head-to-head configuration.

6. The silk polypeptide of claim 1 comprising at least about 2 to about 4 repetitive units.

7. The silk polypeptide of claim 1 comprising at least about 5 to about 10 repetitive units.

8. The silk polypeptide of claim 1 comprising at least about 10 to about 50 repetitive units.

9. The silk polypeptide of claim 1 comprising at least about 50 to about 100 repetitive units.

10. The silk polypeptide of claim 1, wherein at least two of the repetitive units are contiguous.

11. The silk polypeptide of claim 10, wherein the repetitive units are contiguous.

12. The silk polypeptide of claim 1, wherein at least two of the repetitive units are separated by an amino acid spacer.

13. The silk polypeptide of claim 12, wherein the repetitive units are separated from each other by an amino acid spacer.

14. The silk polypeptide of claim 12, wherein the amino acid spacer is 1 to about 10 amino acids in length.

15. The silk polypeptide of claim 1, wherein the repetitive units comprise amino acid sequences forming a secondary structure selected from the group consisting of: β turn spiral, crystalline β sheet, and 3₁₀ helix.

16. The silk polypeptide of claim 1, wherein a repetitive unit comprises a repetitive unit found within an spider or insect silk polypeptide.

17. The silk polypeptide of claim 1, wherein each repetitive unit independently comprises a repetitive unit found within Nephila clavipes or Araneus diadematus spider silk polypeptides or Bombyx mori cocoon silk polypeptides.

18. The silk polypeptide of claim 1, wherein the repetitive units comprise iterated peptide motifs selected from the group consisting of the amino acid sequences identified as SEQ ID NOS:4-27.

19. The silk polypeptide of claim 1, wherein the amino acid sequence of each repetitive unit is independently selected from the amino acid sequences of repetitive units found within the group consisting of ADF-1, ADF-2, ADF-3, ADF-4, ABF-1, MaSpI, MaSpII, MiSpI, MiSpII, and Flag.

20. The silk polypeptide of claim 19, wherein the amino acid sequence of each repetitive unit is selected from the group of amino acid sequences identified as SEQ ID No:1, SEQ ID No:2, and SEQ ID No:3.

21. The silk polypeptide of claim 19, wherein at least one of the native repetitive regions has an amino acid sequence that is in a reversed order in comparison to the naturally-occurring amino terminus to carboxyl terminus amino acid sequence.

22. The silk polypeptide of claim 1, wherein the repetitive units comprise a plurality of iterated peptide motifs selected from the group consisting of: GPG(X)ₙ, (GA)ₙ, Aₙ, and GGX,
where X represents the amino acid A, Q, G, L, S, Y or V, and
n represents an integer from 1 to about 8.

23. The silk polypeptide of claim 1, wherein at least two of the repetitive units have identical amino acid sequences.

24. The silk polypeptide of claim 1, wherein the repetitive units have non-identical amino acid sequences.

25. The silk polypeptide of claim 1, wherein the non-repetitive hydrophilic amino acid domain is towards the carboxyl terminus with respect to the repetitive units.

26. The silk polypeptide of claim 1, wherein the non-repetitive hydrophilic amino acid domain is towards the amino terminus with respect to the repetitive units.

27. The silk polypeptide of claim 1, wherein the non-repetitive hydrophilic amino acid domain is between two of the repetitive units.

28. The silk polypeptide of claim 27, further comprising a proteolytic site, wherein cleavage at the proteolytic site separates a non-repetitive hydrophilic amino acid domain from a repetitive unit.

29. The silk polypeptide of claim 27, further comprising a first proteolytic site and a second proteolytic site, wherein cleavage at the first proteolytic site and at the second proteolytic site separates the non-repetitive hydrophilic amino acid domain from the repetitive units.

30. The silk polypeptide of claim 1, further comprising a plurality of non-repetitive hydrophilic amino acid domains wherein the plurality is at least about 2 to about 4 non-repetitive hydrophilic amino acid domains.

31. The silk polypeptide of claim 1, wherein the non-repetitive hydrophilic amino acid domain is selected from the group consisting of non-repetitive carboxyl terminal regions from MaSpI, MaSpII, ABF-1, ADF-1, ADF-2, ADF-3, ADF-4, and Flag.

32. The silk polypeptide of claim 1, wherein the non-repetitive hydrophilic amino acid domain is about 20 to about 150 amino acids.

33. The silk polypeptide of claim 1 further comprising a proteolytic site, wherein cleavage at the proteolytic site results in the separation of the non-repetitive hydrophilic amino acid domain from a repetitive unit.

34. The silk polypeptide of claim 1 further comprising a proteolytic site, wherein cleavage at the proteolytic site results in the separation of the non-repetitive hydrophilic amino acid domain from the repetitive units.

35. The silk polypeptide of claim 34, wherein the proteolytic site is subject to cleavage by a protease.

36. The silk polypeptide of claim 34, wherein the proteolytic site is subject to cleavage by chemical treatment.

37. The silk polypeptide of claim 1 further comprising a secretory signal peptide sequence.

38. The silk polypeptide of claim 1 further comprising a c-myc epitope.

39. The silk polypeptide of claim 1 further comprising a histidine tag.

40. The silk polypeptide of claim 1, wherein the silk polypeptide has a molecular weight between about 16,000 daltons and about 800,000 daltons.

41. The silk polypeptide of claim 1 wherein the silk polypeptide precipitates and redissolves in an aqueous buffer.

42.-89. (canceled)


SEQUENCE LISTING (48 sequences)

SEQ ID NO: 1 - protein, 646 aa, Artificial sequence
  MaSpI polypeptide
QGAGAAAAAAAGGAGQGGYGGLGSQGAGRGGQGAGAAAAAAGGAGQGGYGGLGSQGAGRGGLGGQGAGAAAAAAAGGVGQGGLGGQGAGQGAGAAAAAAGGAGQGGYGGLGSQGAGRGGSGGQGAGAAAAAAGGAGQGGYGGLGSQGAGRGGLGGQGAGAAAAAAAGGAGQGGYGGLGGQGAGQGGYGGLGSQGAGRGGLGGQGAGAAAAAAAGGAGQGGLGGQGAGQGAGAAAAAAGGAGQGGYGGLGSQGAGRGGQGAGAAAAAAVGAGQGGYGGQGAGQGGYGGLGSQGAGRGGLGGQGAGAAAAAAAGGAGQGGLGGQGAGQGAGAAAAAAGGAGQGGYGGLGNQGAGRGGQGAAAAAAGGAGQGGYGGLGSQGAGRGGLGGQGAGAAAAAAGGAGQGGYGGLGGQGAGQGGYGGLGSQGSGRGGLGGQGAGAAAAAAGGAGQGGLGGQGAGQGAGAAAAAAGGVRQGGYGGLGSQGAGRGGQGAGAAAAAAGGAGQGGYGGLGGQGVGRGGLGGQGAGAAAAGGAGQGGYGGVGSGASAASAAASRLSSPQASSRVSSAVSNLVASGPTNSAALSSTISNVVSQIGASNPGLSGCDCLIQALLEVVSALIQILGSSSIGQCNYGSAGQATQIVGQSVYQAL

SEQ ID NO: 2 - protein, 627 aa, Artificial sequence
  MaSpII polypeptide
PGGYGPGQQGPGGYGPGQQGPSGPGSAAAAAAAAAAGPGGYGPGQQGPGGYGPGQQGPGGYGPGQQGPSGPGSAAAAAAGSGQQGPGGYGPGQQGPGGYGPGQQGPSGPGSAAAASAAASAESGQQGPGGYGPGQQGPGGYGPGQQGPGGYGPGQQGPSGPGSAAAAAAAASGPGQQGPGGYGPGQQGPGGYGPGQQGPSGPGSAAAAAAAASGPGQQGPGGYGPGQQGPGGYGPGQQGLSGPGSAAAAAAAGPGQQGPGGYGPGQQGPSGPGSAAAAAAAAAGPGGYGPGQQGPGGYGPGQQGPSGAGSAAAAAAAGPGQQGLGGYGPGQQGPGGYGPGQQGPGGYGPGSASAAAAAAGPGQQGPGGYGPGQQGPSGPGSASAAAAAAAAGPGGYGPGQQGPGGYAPGQQGPSGPGSASAAAAAAAAGPGGYGPGQQGPGGYAPGQQGPSGPGSAAAAAAAAAGPGGYGPAQQGPSGPGIAASAASAGPGGYGPAQQGPAGYGPGSAVAASAGAGSAGYGPGSQASAAASRLASPDSGARVASAVSNLVSSGPTSSAALSSVISNAVSQIGASNPGLSGCDVLIQALLEIVSACVTILSSSSIGQVNYGAASQFAQVVGQSVLSAF

SEQ ID NO: 3 - protein, 625 aa, Artificial sequence
  ADF-3 polypeptide
GSGQQGPGQQGPGQQGPGQQGPYGPGASAAAAAAGGYGPGSGQQGPSQQGPGQQGPGGQGRYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGQQGPGQQGPGQQGPGGQGPYGPGAASAAVSVGGYGPGSSSVPVASAVASRLSSPAASSRVSSAVSSLVSSGPTKHALLSNTISSVVSQVSANPGLSGCDVLVQALLEVVSALVSILGSSSIGQINYGASAQYTQMVGQSVAQALA

SEQ ID NO: 4 - protein, 5 aa, Artificial sequence
  Acceptable repetitive units of silk polypeptide
AAAAA

SEQ ID NO: 5 - protein, 4 aa, Artificial sequence
  Acceptable repetitive units of silk polypeptide
GAGA

SEQ ID NO: 6 - protein, 6 aa, Artificial sequence
  Acceptable repetitive units of silk polypeptide
GAGAGA

SEQ ID NO: 7 - protein, 8 aa, Artificial sequence
  Acceptable repetitive units of silk polypeptide
GAGAGAGA

SEQ ID NO: 8 - protein, 10 aa, Artificial sequence
  Acceptable repetitive units of silk polypeptide
GAGAGAGAGA

SEQ ID NO: 9 - protein, 12 aa, Artificial sequence
  Acceptable repetitive units of silk polypeptide
GAGAGAGAGAGA

SEQ ID NO: 10 - protein, 14 aa, Artificial sequence
  Acceptable repetitive units of silk polypeptide
GAGAGAGAGAGAGA

SEQ ID NO: 11 - protein, 7 aa, Artificial sequence
  Acceptable repetitive units of silk polypeptide
GGYGQGY

SEQ ID NO: 12 - protein, 8 aa, Artificial sequence
  Acceptable repetitive units of silk polypeptide
AAAAAAAA

SEQ ID NO: 13 - protein, 8 aa, Artificial sequence
  Acceptable repetitive units of silk polypeptide
GGAGQGGY

SEQ ID NO: 14 - protein, 17 aa, Artificial sequence
  Acceptable repetitive units of silk polypeptide
GGQGGQGGYGGLGSQGA

SEQ ID NO: 15 - protein, 8 aa, Artificial sequence
  Acceptable repetitive units of silk polypeptide
ASAAAAAA

SEQ ID NO: 16 - protein, 5 aa, Artificial sequence
  Acceptable repetitive units of silk polypeptide
GPGQQ

SEQ ID NO: 17 - protein, 10 aa, Artificial sequence
  Acceptable repetitive units of silk polypeptide
GPGQQGPGQQ

SEQ ID NO: 18 - protein, 15 aa, Artificial sequence
  Acceptable repetitive units of silk polypeptide
GPGQQGPGQQGPGQQ

SEQ ID NO: 19 - protein, 20 aa, Artificial sequence
  Acceptable repetitive units of silk polypeptide
GPGQQGPGQQGPGQQGPGQQ

SEQ ID NO: 20 - protein, 25 aa, Artificial sequence
  Acceptable repetitive units of silk polypeptide
GPGQQGPGQQGPGQQGPGQQGPGQQ

SEQ ID NO: 21 - protein, 30 aa, Artificial sequence
  Acceptable repetitive units of silk polypeptide
GPGQQGPGQQGPGQQGPGQQGPGQQGPGQQ

SEQ ID NO: 22 - protein, 35 aa, Artificial sequence
  Acceptable repetitive units of silk polypeptide
GPGQQGPGQQGPGQQGPGQQGPGQQGPGQQGPGQQ

SEQ ID NO: 23 - protein, 40 aa, Artificial sequence
  Acceptable repetitive units of silk polypeptide
GPGQQGPGQQGPGQQGPGQQGPGQQGPGQQGPGQQGPGQQ

SEQ ID NO: 24 - protein, 12 aa, Artificial sequence
  Acceptable repetitive units of silk polypeptide
GPGGQGGPYGPG

SEQ ID NO: 25 - protein, 10 aa, Artificial sequence
  Acceptable repetitive units of silk polypeptide
SSAAAAAAAA

SEQ ID NO: 26 - protein, 8 aa, Artificial sequence
  Acceptable repetitive units of silk polypeptide
GPGSQGPS

SEQ ID NO: 27 - protein, 5 aa, Artificial sequence
  Acceptable repetitive units of silk polypeptide
GPGGY

SEQ ID NO: 28 - protein, 34 aa, Nephila spidroin
AGQGGYGGLGSQGAGRGGLGGQGAGAAAAAAAGG

SEQ ID NO: 29 - protein, 47 aa, Nephila spidroin
CPGGYGPGQQCPGGYGPGQQCPGGYGPGQQGPSGPGSAAAAAAAAAA

SEQ ID NO: 30 - DNA, 30 bp, Artificial sequence
  Primer
cgtacgaagcttatgcacgagccggatctg

SEQ ID NO: 31 - DNA, 33 bp, Artificial sequence
  Primer
attaactcgagcagcaagggcttgagctacaga

SEQ ID NO: 32 - DNA, 15 bp, Artificial sequence
  Linker sequence
tcgagcttgatgttt

SEQ ID NO: 33 - DNA, 157 bp, Artificial sequence
  Linker sequence
caggatctggacaacaaggacccggacaacaaggacccggacaacaaggacccggacaacaaggaccatatggacccggtgcatccgccgcagcagcagccgctggaggttatggacccggatctggacaacaaggacccagccaacaaggacctgg

SEQ ID NO: 34 - DNA, 18 bp, Artificial sequence
  Linker sequence
ctaggttaagtttaaacg

SEQ ID NO: 35 - DNA, 59 bp, Artificial sequence
  Primer
caggttccactggtgacgcggcccaaggggcccaaggggcaggtgcagcagcagcagca

SEQ ID NO: 36 - DNA, 25 bp, Artificial sequence
  Primer
gaacccagagcagcagtacccatag

SEQ ID NO: 37 - DNA, 16 bp, Artificial sequence
  Linker sequence
agcgggcccgctcttc

SEQ ID NO: 38 - DNA, 13 bp, Artificial sequence
  Primer
gaagagcgggccc

SEQ ID NO: 39 - DNA, 17 bp, Artificial sequence
  Linker sequence
gggctgctgctgcggcc

SEQ ID NO: 40 - DNA, 17 bp, Artificial sequence
  Primer
gggctgctgctgcggcc

SEQ ID NO: 41 - DNA, 10 bp, Artificial sequence
  Linker sequence
tgaaatttcg

SEQ ID NO: 42 - DNA, 18 bp, Artificial sequence
  Primer
aattcgaaatttcatgca

SEQ ID NO: 43 - protein, 6 aa, Artificial sequence
  Crystal forming Gly-rich amorphous blocks of spider silk protein
GGYGPG

SEQ ID NO: 44 - protein, 16 aa, Artificial sequence
  Anti-MaSpII sequence
GLGSQGAGRGGQGAGA

SEQ ID NO: 45 - protein, 16 aa, Artificial sequence
  Anti-ADF-3 sequence
ARAGSGQQGPGQQGPG

SEQ ID NO: 46 - protein, 360 aa, Artificial sequence
  Translation of ADF-1
HESSYAAAMAASTRNSDFIRNMSYQMGRLLSNAGAITESTASSAASSASSTVTESIRTYGPAAIFSGAGAGAGVGVGGAGGYGQGYGAGAGAGAGAGAGAGGAGGYGQGYGAGAAAAAGAGAGAAGGYGGGSGAGAGGAGGYGQGYGAGSGAGAGAAAAAGASAGAAGGYGGGAGVGAGAGAGAAGGYGQSYGSGAGAGAGAGAAAAAGAGARAAGGYGGGYGAGAGAGAGAAASAGASGGYGGGYGGGAGAGAVAGASAGSYGGAVNRLSSAGAASRVSSNVAAIASAGAAALPNVISNIYSGVLSSGVSSSEALIQALLEVISALIHVLGSASIGNVSSVGVNSALNAVQNAVGAYAG

SEQ ID NO: 47 - protein, 294 aa, Artificial sequence
  Translation of ADF-2
GSQGAGGAGQGGYGAGGGGAAAAAAAAVGAGGGGQGGLGSGGAGQGYGAGLGGQGGASAAAAAAGGQGGQGGQGGYGGLGSQGAGGAGQLGYGAGQESAAAAAAAAGGAGGGGQGGLGAGGAGQGYGAAGLGGQGGAGQGGGSGAAAAAGGQGGQGGYGGLGPQGAGGAGQGGYGGGSLQYGGQGQAQAAAASAAASRLSSPSAAARVSSAVSLVSNGGPTSPAALSSSISNVVSQISASNPGLSGCDILVQALLEIISALVHILGSANIGPVNSSSAGQSASIVGQSVYRALS

SEQ ID NO: 48 - protein, 410 aa, Artificial sequence
  Translation of ADF-4
AGSSAAAAAAASGSGGYGPENQGPSGPVAYGPGGPVSSAAAAAAAGSGPGGYGPENQGPSGPGGYGPGGSGSSAAAAAAAASGPGGYGPGSQGPSGPGGSGGYGPGSQGASGPGGPGASAAAAAAAAAASGPGGYGPGSQGPSGPGAYGPGGPGSSAAAAAAAASGPGGYGPGSQGPSGPGVYGPGGPGSSAAAAAAAGSGPGGYGPENQGPSGPGGYGPGGSGSSAAAAAAAASGPGGYGPGSQGPSGPGGSGGYGPGSQGGSGPGASAAAAAAAASGPGGYGPGSQGPSGPGYQGPSGPGAYGPSPSASASVAASVYLRLQPRLEVSSAVSSLVSSGPTNGAAVSGALNSLVSQISASNPGLSGCDALVQALLELVSALVAILSSASIGQVNVSSVSQSTQMISQALS